(19) United States
(12) Patent Application Publication
(10) Pub. No.: US 2020/0038514 A1
(43) Pub. Date: Feb. 6, 2020

(54) FOLATE CONJUGATE FOR USE IN TARGETING TUMOR ASSOCIATED MACROPHAGES

(71) Applicant: ENDOCYTE, INC., West Lafayette, IN (US)

(72) Inventors: Iontcho Radoslavov VLAHOV, West Lafayette, IN (US); Christopher Paul LEAMON, West Lafayette, IN (US); Longwu QI, San Mateo, CA (US); Ning ZOU, West Lafayette, IN (US); Kevin Yu WANG, Zionsville, IN (US); Albert E. FELTEN, Lafayette, IN (US); Garth L. PARHAM, Largo, FL (US); Fei YOU, Rockville, MD (US); Hari Krishna R. SANTHAPURAM, West Lafayette, IN (US); Spencer J. HAHN, West Lafayette, IN (US); Joseph Anand REDDY, West Lafayette, IN (US); Yingjuan J. LU, West Lafayette, IN (US); Leroy W. WHEELER, II, West Lafayette, IN (US)

(21) Appl. No.: 16/498,071

(22) PCT Filed: Sep. 14, 2017
(86) PCT No.: PCT/US2017/051662

§ 371 Date: Sep. 26, 2019

Related U.S. Application Data
(63) Continuation-in-part of application No. PCT/US17/24770, filed on Mar. 29, 2017.
(60) Provisional application No. 62/314,688, filed on Mar. 29, 2016.
(60) Provisional application No. 62/323,282, filed on Apr. 15, 2016.
(60) Provisional application No. 62/396,409, filed on Sep. 19, 2016.

(51) Int. Cl. A61K 47/55; A61P 35/00

(52) U.S. Cl. CPC A61K 47/551; A61P 35/00

(57) ABSTRACT

Methods are provided for treating cancers using a conjugate herein described as Conjugate 5, or a pharmaceutically acceptable salt thereof. Methods for treating cancers using Conjugate 5, or a pharmaceutically acceptable salt thereof, to target tumor associated macrophages are also described.

### CROSS REFERENCE TO RELATED APPLICATIONS

This application claims priority to PCT International Application No. PCT/US2017/024770 filed Mar. 29, 2017, which is incorporated herein by reference in its entirety.

### FIELD OF THE INVENTION

The invention described herein relates to methods for treating cancers using a conjugate having the formula

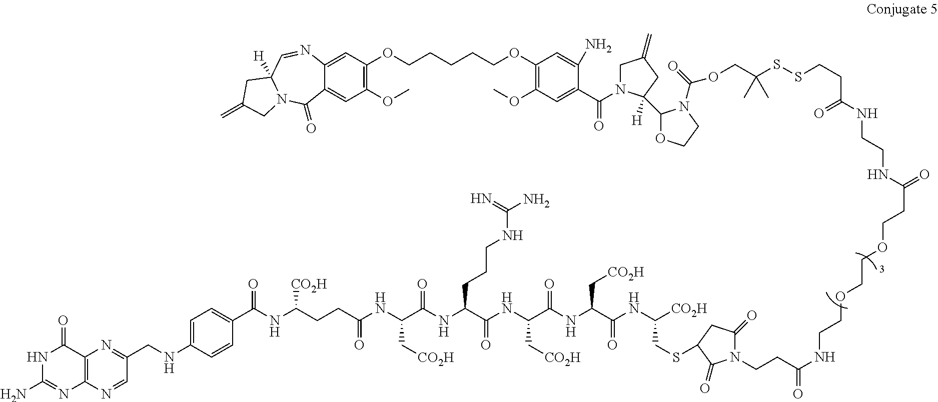

(hereinafter referred to as “Conjugate 5”). The invention described herein also relates to methods for treating cancers using Conjugate 5 to target tumor associated macrophages.

### BACKGROUND AND SUMMARY OF THE INVENTION

Despite the fact that there have been significant developments in anti-cancer technology, such as radiotherapy, chemotherapy and hormone therapy, cancer still remains the second leading cause of death following heart disease in the United States. Most often, cancer is treated with chemotherapy utilizing highly potent drugs, such as mitomycin, paclitaxel and camptothecin. In many cases, these chemotherapeutic agents show a dose responsive effect, and cell killing is proportional to drug dose. A highly aggressive style of dosing is thus necessary to eradicate neoplasms; however, high-dose chemotherapy is hindered by poor selectivity for cancer cells and severe toxicity to normal cells. This lack of tumor-specific treatment is one of the many hurdles that needs to be overcome by current chemotherapy.

One solution to current chemotherapy limitations is to deliver a biologically effective concentration of an agent to tumor tissue with very high specificity. To reach this goal, much effort has been undertaken to develop tumor-selective drugs by conjugating anti-cancer drugs to hormones, antibodies, and vitamins. For example, the low molecular weight vitamin, folic acid, and other folate receptor binding compounds and ligands are especially useful as targeting agents for folate receptor-positive cancer cells and tumors.

Folic acid is a member of the B family of vitamins and plays an essential role in cell survival by participating in the biosynthesis of nucleic and amino acids. This essential vitamin is also a high affinity ligand that enhances the specificity of conjugated anti-cancer drugs by targeting folate receptor-positive cancer cells. It has been found that the folate receptor (FR) is up-regulated in more than 90% of non-mucinous ovarian carcinomas. The folate receptor is also found at high to moderate levels in kidney, brain, lung, and breast carcinomas. At the same time, it has been reported that the folate receptor occurs at low levels in most normal tissues leading to a mechanism for selectively targeting the cancer cells. Although the folate receptor can be used to deliver agents to tumor tissue with very high specificity, there are a number of cancers that do not express the folate receptor at all, or not in sufficient numbers to provide the desired specificity. Thus, there is a need for developing targeted therapies to deliver agents to such folate receptor negative cancers.

Tumor-associated macrophages (TAMs) exist that are pro-tumorigenic. These macrophages are found in the tumor microenvironment, and can be pro-tumorigenic by causing such responses as inhibition of B and T cell activation, inhibition of tumor-associated antigen presentation, inhibition of cytotoxic granule release, and increased angiogenesis. Thus, therapies that deplete TAMs or inhibit their activity would be useful.

Applicants have discovered that tumors and cancers that either overexpress the folate receptor or do not express the folate receptor in sufficient numbers, or at all, can be treated by targeting drugs to TAMs. Described herein are methods for treating cancers by targeting TAMs using Conjugate 5, or a pharmaceutically acceptable salt thereof, as a TAM-targeting agent. Applicants have discovered that a subset of TAMs that is pro-tumorigenic expresses the folate receptor (3, also known as folate receptor 2. Thus, Applicants have discovered that these pro-tumorigenic TAMs can be targeted using folate as the targeting ligand to deliver the conjugate to these TAMs to deplete or inhibit the pro-tumorigenic TAMs to treat cancer in a host animal whether or not the cancer cells themselves express folate receptors. It is to be understood that the methods described herein can be used to treat cancers that do not express the folate receptor, as well as cancers that do express the folate receptor.

In one embodiment, a method for treating a cancer is provided. The method comprises the steps of identifying the presence of tumor-associated macrophages in a cancer in a host animal, and administering to the host animal a therapeutically effective amount of Conjugate 5, or a pharmaceutically acceptable salt thereof.

In another embodiment, a method for treating a cancer is provided. The method comprises the step of administering to the host animal a therapeutically effective amount of Conjugate 5, or a pharmaceutically acceptable salt thereof, wherein the host animal has previously been administered a folate imaging agent conjugate and the host animal's folate receptor status has been determined to be negative.

In another embodiment, a method for treating a cancer in a host animal by inhibiting or depleting tumor-associated macrophages in the host animal is provided. The method comprises the step of administering to the host animal a therapeutically effective amount of Conjugate 5, or a pharmaceutically acceptable salt thereof, wherein the tumor-associated macrophages are inhibited or depleted.

In another embodiment, a method for targeting tumor-associated macrophages in a host animal is provided. The method comprises the step of administering to the host animal a therapeutically effective amount of Conjugate 5, or a pharmaceutically acceptable salt thereof, wherein the tumor-associated macrophages are targeted.

In another embodiment, a method for treating a cancer in a host animal where tumor-associated macrophages are part of the cancer, tissue, or tumor is provided. The method comprises the steps of administering to the host animal a therapeutically effective amount of Conjugate 5, or a pharmaceutically acceptable salt thereof, and treating the cancer having the tumor-associated macrophages. In one embodiment Conjugate 5, or a pharmaceutically acceptable salt thereof, includes a folate that binds to the folate receptor-α and/or the folate receptor-β.

In another embodiment, a method for treating a folate receptor negative cancer is provided. The method comprises administering to the host animal a therapeutically effective amount of Conjugate 5, or a pharmaceutically acceptable salt thereof, wherein tumor-associated macrophages are inhibited or depleted.

In another embodiment, a method for treating a folate receptor negative cancer is provided. The method comprises administering to the host animal a therapeutically effective amount of Conjugate 5, or a pharmaceutically acceptable salt thereof, to deplete tumor-associated macrophages.

In another embodiment, a method for treating a folate receptor negative cancer is provided. The method comprises administering to the host animal a therapeutically effective amount of Conjugate 5, or a pharmaceutically acceptable salt thereof, and treating the folate receptor negative cancer having tumor-associated macrophages.

In another embodiment, a method for treating a folate receptor negative cancer in a host animal is provided. The method comprises administering to the host animal a therapeutically effective amount of Conjugate 5, or a pharmaceutically acceptable salt thereof, to target tumor associated macrophages.

Additional illustrative and non-limiting embodiments of the invention are described in the following enumerated clauses. All combinations of the following clauses are understood to be additional embodiments of the invention described herein.

1. A method for treating a folate receptor negative cancer comprising administering to the host animal a therapeutically effective amount of Conjugate 5, or a pharmaceutically acceptable salt thereof, wherein tumor-associated macrophages are inhibited or depleted.

2. A method for treating a folate receptor negative cancer comprising administering to the host animal a therapeutically effective amount of Conjugate 5, or a pharmaceutically acceptable salt thereof, to deplete tumor-associated macrophages.

3. A method for treating a folate receptor negative cancer comprising administering to the host animal a therapeutically effective amount of Conjugate 5, or a pharmaceutically acceptable salt thereof, and treating the folate receptor negative cancer having tumor-associated macrophages.

4. A method for treating a folate receptor negative cancer in a host animal comprising administering to the host animal a therapeutically effective amount of Conjugate 5, or a pharmaceutically acceptable salt thereof, to target tumor associated macrophages.

5. A method for treating a cancer comprising the steps of identifying the presence of tumor-associated macrophages in the cancer in a host animal, and administering to the host animal a therapeutically effective amount of Conjugate 5, or a pharmaceutically acceptable salt thereof.

6. A method for treating a cancer in a host animal, the method comprising the step of administering to the host animal a therapeutically effective amount of Conjugate 5, or a pharmaceutically acceptable salt thereof, to inhibit or deplete tumor-associated macrophages in the host animal.

7. A method for targeting tumor-associated macrophages in a host animal, the method comprising the step of administering to the host animal a therapeutically effective amount of Conjugate 5, or a pharmaceutically acceptable salt thereof, to target the tumor-associated macrophages.

8. A method for treating a cancer in a host animal where tumor-associated macrophages are in the cancer and/or form part of the tissue or tumor, the method comprising the steps of administering to the host animal a therapeutically effective amount of Conjugate 5, or a pharmaceutically acceptable salt thereof, and treating the cancer having the tumor-associated macrophages.

9. The method of any one of clauses 1 to 8 wherein tumor associated macrophages are in the cancer and the tumor-associated macrophages have the pro-tumor M2-biased CD163(+) phenotype.

10. The method of any one of clauses 1 to 8 wherein tumor-associated macrophages are in the cancer and the tumor-associated macrophages have the pro-tumor M2-biased CD163(+) and TGF-β(+) phenotype.

11. The method of any one of clauses 1 to 8 wherein tumor-associated macrophages are in the cancer and the tumor-associated macrophages have the pro-tumor M2-biased CD11b(+) phenotype.

12. The method of any one of clauses 1 to 8 wherein tumor-associated macrophages are in the cancer and the tumor-associated macrophages have the pro-tumor M2-biased CD163(+) and CD11b(+) phenotype.

13. The method of any one of clauses 1 to 8 wherein tumor-associated macrophages are in the cancer and the tumor-associated macrophages have the pro-tumor M2-biased F480(+) phenotype.

14. The method of any one of clauses 1 to 8 wherein tumor-associated macrophages are in the cancer and the tumor-associated macrophages have the pro-tumor M2-biased F480(+) and CD11b(+) phenotype.

15. The method of any one of clauses 1 to 8 wherein the tumor-associated macrophages are in the cancer and the tumor-associated macrophages are pro-tumor M2-biased and express one or more markers selected from the group consisting of CD163(+), IL10(+), Arg1(+), TGF-β(+), VEGF(+), CD206(+), CD11b(+), and F480(+) phenotype.

16. The method of any one of clauses 1 to 8 wherein tumor-associated macrophages are in the cancer and/or form part of the tissue or cancer and the tumor-associated macrophages are pro-tumor M2-biased and express one or more markers selected from the group consisting of CD163(+), IL10(+), Arg1(+), TGF-β(+), VEGF(+), CD206(+), CD11b(+), and F480(+) phenotype.

17. The method of any one of clauses 1 to 16 wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkin's lymphoma, uveal melanoma, glioblastoma, renal carcinoma, leiomyosarcoma, and pigmented villonodular synovitis.

18. The method of any one of clauses 1 to 17 wherein Conjugate 5, or a pharmaceutically acceptable salt thereof, is capable of depleting, or depletes the tumor-associated macrophages in the host animal.

19. The method of any one of clauses 1 to 18 wherein Conjugate 5, or a pharmaceutically acceptable salt thereof, is capable of inhibiting, or inhibits the activity of the tumor-associated macrophages in the host animal.

20. The method of any one of clauses 1 to 19 wherein Conjugate 5, or a pharmaceutically acceptable salt thereof, is administered to the host animal in a parenteral dosage form.

21. The method of clause 20 wherein the parenteral dosage form is selected from the group consisting of intradermal, subcutaneous, intramuscular, intraperitoneal, intravenous, and intrathecal dosage forms.

22. The method of any one of clauses 1 to 21 wherein the therapeutically effective amount is from about 0.05 μmol/kg to about 6.0 μmol/kg of host animal body weight.

23. The method of any one of clauses 1 to 22 wherein the therapeutically effective amount is from about 0.05 μmol/kg to about 4.0 μmol/kg of host animal body weight.

24. The method of any one of clauses 1 to 23 wherein the therapeutically effective amount is from about 0.05 μmol/kg to about 2.0 μmol/kg of host animal body weight.

25. The method of any one of clauses 1 to 24 wherein the therapeutically effective amount is from about 0.05 μmol/kg to about 1.0 μmol/kg of host animal body weight.

26. The method of any one of clauses 1 to 8 wherein tumor-associated macrophages are in the cancer and the tumor-associated macrophages have the pro-tumor M2-biased CD163(+), IL10(+), Arg1(+), TGF-β(+), VEGF(+), CD206(+), CD11b(+), and F480(+) phenotype.

In any of the embodiments described herein, the cancer may express folate receptors, or may not express folate receptors. In any of the embodiments in the preceding paragraphs tumor associated macrophages are in the cancer and the tumor-associated macrophages may have the pro-tumor M2-biased CD163(+) phenotype, the pro-tumor M2-biased CD163(+) and TGF-β(+) phenotype, the pro-tumor M2-biased CD163(+), IL10(+), Arg1(+), TGF-β(+), VEGF(+), and CD206(+) phenotype, or the tumor-associated macrophages are pro-tumor M2-biased and may express one or more markers selected from the group consisting of CD163(+), IL10(+), Arg1(+), TGF-β(+), VEGF(+), CD206(+), CD11b(+), F480(+), CD163(+)CD11b(+), and F480(+)CD11b(+).

In any of the embodiments described herein, the cancer can be selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis.

In any of the embodiments described herein, Conjugate 5 or a pharmaceutically acceptable salt thereof, can be administered to the host animal in a parenteral dosage form. The parenteral dosage form can be selected from the group consisting of intradermal, subcutaneous, intramuscular, intraperitoneal, intravenous, and intrathecal. In any of the embodiments described herein, the therapeutically effective amount can be from about 0.1 μmol/kg to about 6.0 μmol/kg of Conjugate 5, or a pharmaceutically acceptable salt thereof; from about 0.1 μmol/kg to about 4.0 μmol/kg of Conjugate 5, or a pharmaceutically acceptable salt thereof; or from about 0.1 μmol/kg to about 2.0 μmol/kg of Conjugate 5, or a pharmaceutically acceptable salt thereof.

### BRIEF DESCRIPTION OF THE DRAWINGS

FIG. 1 is a chart that shows the percentage of ³H-thymidine incorporated into KB cells treated with Conjugate 5 (●) and with Conjugate 5 and excess folate (▪).

FIG. 2A is a chart that shows that Conjugate 5 dosed at 0.5 μmol/kg SIW for two weeks (▴) decreased KB tumor size in test mice compared to untreated control (▪). The dotted line indicates the last dosing day.

FIG. 2B is a chart that shows % weight change for test mice dosed at 0.5 μmol/kg Conjugate 5 SIW for two weeks (▴) compared to untreated control (▪).

FIG. 3 is a chart showing that mice bearing paclitaxel resistant KB tumors dosed at 0.5 μmol/kg SIW for two weeks with Conjugate 5 (▴) had decreased tumor size compared to untreated control (▪). The dotted line indicates the last dosing day. n=5, Conjugate 5 {0,1,4} as {partial response, complete response, cure}.

FIG. 4 is a chart showing that mice bearing platinum resistant KB tumors dosed at 0.5 μmol/kg SIW for two weeks with Conjugate 5 (▪), and dosed at 2.0 μmol/kg BIW for two weeks with EC1456 (▾) had decreased tumor size compared to untreated control (●). The dotted line indicates the last dosing day. n=4, Conjugate 5 {0,0,4}; EC1446 {0,2,2} as {partial response, complete response, cure}.

FIG. 5 is a chart showing that mice bearing ST502 TNBC PDX tumors dosed at 0.3 μmol/kg BIW for two weeks with Conjugate 5 (▴) had decreased tumor size compared to untreated control (▪), while mice dosed at 2.0 μmol/kg BIW for two weeks with EC1456 (●) did not have decreased tumor size compared to untreated control (▪). The dotted line indicates the last dosing day. n=7, Conjugate 5 {0,0,7} as {partial response, complete response, cure}.

FIG. 6 is a chart showing that mice bearing ST070 ovarian PDX tumors dosed at 0.5 μmol/kg SIW for two weeks with Conjugate 5 (●) had decreased tumor size compared to untreated control (▪), while mice dosed at 4.0 μmol/kg SIW for two weeks with EC1456 (▴) or dosed at 15.0 mg/kg SIW for two weeks with paclitaxel (▾) did not have decreased tumor size. The dotted line indicates the last dosing day. n=7, Conjugate 5 {0,0,7} as {partial response, complete response, cure}.

FIG. 7 is a chart that shows the relative binding affinity of Conjugate 5 toward the folate receptor. The experiment shows that the relative binding affinity of Conjugate 5 was ˜1.9-fold lower than that of folic acid. (▪) folic acid (Control); (●) Conjugate 5.

FIG. 8 is a graph that shows that intact Conjugate 5 is not able to crosslink DNA while the reduced form (treated with DTT) releases the active PBD molecule, which can then crosslink with DNA. (●) Conjugate 5 plus DTT; (▪) Conjugate 1 without DTT.

FIG. 9A is a chart that shows that Conjugate 5 dosed at 0.1 μmol/kg SIW for two weeks (▪) and Conjugate 5 dosed at 0.15 μmol/kg SIW for two weeks (▴) decreased KB tumor size in test rats compared to untreated control (●). The dotted line indicates the last dosing day.

FIG. 9B is a chart that shows % weight change for test rats dosed at 0.1 μmol/kg Conjugate 5 SIW for two weeks (▪) and test mice dosed at 0.15 μmol/kg Conjugate 5 SIW for two weeks (▴) compared to untreated control (●).

FIG. 10 is a chart that shows that Conjugate 5 dosed at 0.27 μmol/kg BIW for two weeks (●) decreased TNBC PDX tumor size in test mice compared to untreated control (▪), whereas erubulin mesylate dosed at 1.0 μmol/kg SIW for two weeks (▴) did not decrease TNBC PDX tumor size.

FIG. 11 is a chart that shows that Conjugate 5 dosed at 0.27 μmol/kg BIW for two weeks (●) produced partial response in Endometrial PDX tumor size in test mice compared to untreated control (▪), whereas paclitaxel dosed at 15.0 mg/kg SIW for two weeks (▴) did not produce a partial response.

FIG. 12 is a chart showing a potent dose-dependent inhibition of cell proliferation with relative IC₅₀ values of ˜0.52 (72 h), 0.61 (96 h), and 0.17 (120 h) in ID8-CI15 ovarian cancer cells treated with Conjugate 5.

FIG. 13 is a graph showing that Conjugate 5 demonstrated a potent activity at all concentrations tested (1 nM, 10 nM and 100 nM) after a 2 hour exposure and 9-day chase. The anti-tumor activity of Conjugate 5 was significantly reduced in the presence of excess amount of folic acid at both 1 nM and 10 nM concentrations.

FIG. 14 is a graph showing functional FR levels were measured on the IGROV1 human ovarian cancer cells: (a) hHLA+CD45-ascites cancer cells [FR+=6.04%; (b) ascites F480+CD11+ macs [FR+=52.6%]; (c) IGROV cell line control [FR+=98.5%].

FIG. 15A is chart showing the presence of CD4+ and CD8+ T cells quantitated in total peritoneal cells of the immunocompetent C57BL6 mice at 7 day intervals post IP injection of the mouse ovarian cell line, ID8-CL15 (FIG. 15A). The CD45+CD3e+CD8+CD4− T cells (▪) slowly increased in number from day 7 to day 42 post implantation. The CD45+CD3e+CD4+CD8− T cells (▴) also increased in number from day 7 to day 35.

FIG. 15B is a chart showing CD45-non bone-marrow derived ascites cells from ID8-CL15 implanted mice expressed very little functional FR (see FIG. 15B (▪)), whereas ascites macrophages expressed a significant amount of a functional FR (see FIG. 15B (●)).

FIG. 15C is a graph showing ascites macrophages expressed a significant amount of a functional FR.

FIG. 16A is a chart that shows that Conjugate 5 dosed at 100 nmol/kg BIW, 6 doses, first dose at day 7 (▴) increased survival time in test mice compared to untreated control (●) and anti-CTLA-5 alone dosed at 250 μg/dose BIW, 5 doses, and comparable to a significantly higher dose of comparator compound EC1456 (▾) 2000 nmol/kg BIW, 6 doses, first dose at day 7. FIG. 16A also shows that Conjugate 5 dosed with anti-CTLA-5, initiated at day 11, (∘) increased survival time in test mice compared to all other test animals. The dotted line indicates the last dosing day.

FIG. 16B is a chart that shows % weight change for test mice dosed with Conjugate 5 (▴), Conjugate 5+anti-CTLA-5 (▪), EC1456 (▾) and anti-CTLA-5 (∘) compared to untreated control (●).

FIG. 17A is a chart that shows Conjugate 5 dosed at 0.1 mol/kg, BIW×3, 6 doses, first dose at 7 days (∘) increased survival time in test mice compared to significantly higher dose of comparator compound EC1456 dosed at 2 mol/kg, BIW×3, 6 doses, first dose at 7 days (▾) and untreated control (●).

FIG. 17B is a chart that shows % weight change for test mice dosed with Conjugate 5 (∘), EC1456 (▾), and an untreated control (●) as described in 1A.

FIG. 18A is a chart that shows Conjugate 5 dosed at 0.1 mol/kg, DO-2×3, n=5 mice (animals displayed mild ataxia), first dose at 21 days (∘) increased survival time in test mice compared to significantly higher dose of comparator compound EC1456 dosed at 2 mol/kg, DO-2×3, n=2 mice (3 were euthanized on day 44 due to sever dermatitis) first dose at 21 days (▾), and untreated control (▪).

FIG. 18B is a chart that shows % weight change for test mice dosed with Conjugate 5 (∘), EC1456 (▾), and an untreated control (●) as described in FIG. 18A.

FIG. 19A is a chart that shows Conjugate 5 dosed at 0.3 mol/kg, D35, D42, SIW×2 (∘) increased survival time in test mice compared to significantly higher dose of comparator compound EC1456 dosed at 2 mol/kg, D0-2×2 (▾), and untreated control (●).

FIG. 19B is a chart that shows % weight change for test mice dosed with Conjugate 5 (∘), EC1456 (▾), and an untreated control (▪) as described in FIG. 19A.

FIG. 20A is a chart that shows Conjugate 5 dosed at 0.3 mol/kg, SIW×2 (∘) increased survival time in test mice compared to significantly higher dose of comparator compound EC1456 dosed at 2 mol/kg, D0-2×1 (▾), and untreated control (●).

FIG. 20B is a chart that shows % weight change for test mice dosed with Conjugate 5 (∘), EC1456 (▾), and an untreated control (●) as described in FIG. 20A.

FIG. 21 is a comparison of Conjugate 5 and EC1456 against various stages of ID8-C115 tumor bearing mice.

FIG. 22A is a comparison of Conjugate 5 in-vitro activity against 4T1-C12 tumor cells.

FIG. 22B is comparison of Conjugate 5 in-vitro activity against 4T1p tumor cells.

FIG. 23 is a comparison of Conjugate 5 and EC1456 in-vitro activity against human IGROV Cells after a 2 hour exposure and 9-day chase.

FIG. 24A is an assessment of tumor-associated macrophages in 4T1p and 4T1-C12 Tumors

FIG. 24B shows tumor-associated macrophages found in 4T1p tumors expressed FRβ while other non-macrophage myeloid cells (MDSCs) were FRβ-negative.

FIG. 24C shows tumor-associated macrophages found in 4T1p tumors expressed FRβ while other non-macrophage myeloid cells (MDSCs) were FRβ-negative.

FIG. 25A is a chart showing P-1780 4T1P Balb/c mice tumor volume DOI Apr. 20, 2016 5×10⁵ mammary tumors with Conjugate 5 treatment at 200 nmol/kg (BIW×2) (A) versus an untreated control (B).

FIG. 25B is a chart that shows % weight change for test mice dosed with Conjugate 5 (A) and an untreated control (B) as described in 4A.

FIG. 26A is a chart showing P-1780 4T1P Balb/c mice tumor volume DOI Apr. 7, 2016 5×10⁶ mammary tumors with Conjugate 5 treatment at 200 nmol/kg (BIW×2) (A) versus an untreated control (B).

FIG. 26B is a chart that shows % weight change for test mice dosed with Conjugate 5 (A) and an untreated control (B) as described in 5A.

FIG. 27 contains charts demonstrating apoptotic CD163-CD11b−, CD163-CD11b+, and CD163+CD11b+ when treatment of untreated control (●), Conjugate 5 (▪), Conjugate 5+EC0923 (▴), and EC0923 (▾).

### DETAILED DESCRIPTION OF ILLUSTRATIVE EMBODIMENTS

It is to be understood that each embodiment of the invention described herein may be, as applicable, combined with any other embodiment described herein. For example, any of the embodiments in the Summary, and/or of the enumerated clauses described herein, or any combination thereof, may be combined with any of the embodiments described in the Detailed Description.

Applicants have discovered methods for treating cancers by targeting TAMs (for example, pro-tumor M2-biased TAMs) using Conjugate 5, or a pharmaceutically acceptable salt thereof, as a TAM-targeting agent. Applicants have discovered that a subset of TAMs that is pro-tumorigenic expresses the folate receptor β which is useful for targeting TAMs with Conjugate 5, or a pharmaceutically acceptable salt thereof, using folates as targeting agents. In one embodiment, targeting of the pro-tumorigenic TAMs to deplete TAMs or to inhibit the activity of TAMs can result in inhibition of tumor growth, elimination of a tumor, or stable disease, and like therapeutic effects for the host animal. The methods described herein can be used to treat cancers that do not express the folate receptor, as well as cancers that do express the folate receptor.

In one embodiment, the tumor-associated macrophages described herein are pro-tumor and M2-biased, and, if depleted or inhibited, the host animal's condition may be improved. Such TAMs may have a phenotype resulting from the expression of one or more markers selected from CD163(+), IL10(+), Arg1(+), TGF-β(+), VEGF(+), CD206(+), CD11b(+), F480(+), CD163(+)CD11b(+), F480(+)CD11b(+) and combinations thereof. In another illustrative aspect, the tumor-associated macrophages described herein that are pro-tumor and M2-biased have a CD163(+) phenotype. In yet another embodiment, the tumor-associated macrophages described herein that are pro-tumor and M2-biased have a CD163(+) and TGF-β(+) phenotype. In another embodiment, the tumor-associated macrophages described herein that are pro-tumor and M2-biased have a CD163(+) and CD11b(+) phenotype. In yet another embodiment, the tumor-associated macrophages described herein that are pro-tumor and M2-biased have a F480(+) and CD11b(+) phenotype. In another aspect, the tumor-associated macrophages described herein that are pro-tumor and M2-biased have a phenotype resulting from the expression of CD163(+), IL10(+), Arg1(+), TGF-β(+), VEGF(+), CD206(+), CD11b(+), and F480(+) markers. In another embodiment, the tumor-associated macrophages described herein that are pro-tumor and M2-biased have a phenotype resulting from the expression of one or more markers selected from the group consisting of CD163(+), IL10(+), Arg1(+), TGF-β(+), VEGF(+), and CD206(+), CD11b(+), and F480(+). In one aspect, the presence of the tumor-associated macrophages (e.g., pro-tumor M2-biased TAMs) in the tumor indicates a poor prognosis for the host animal without the therapy described herein.

In one embodiment of the methods described herein for treating a cancer by targeting TAMs, the method comprises the steps of identifying the presence of tumor-associated macrophages (e.g., pro-tumor M2-biased TAMs) in a cancer in a host animal, and administering to the host animal a therapeutically effective amount of Conjugate 5 or a pharmaceutically acceptable salt thereof.

In another embodiment, a method for treating a cancer by targeting TAMs (e.g., pro-tumor M2-biased TAMs) is provided. The method comprises the step of administering to the host animal a therapeutically effective amount of Conjugate 5, or a pharmaceutically acceptable salt thereof, wherein the host animal has previously been administered a folate imaging agent conjugate and the host animal's folate receptor status has been determined to be negative.

In yet another embodiment, a method for treating a cancer in a host animal by inhibiting or depleting tumor-associated macrophages (e.g., pro-tumor M2-biased TAMs) in the host animal is provided. The method comprises the step of administering to the host animal a therapeutically effective amount of Conjugate 5, or a pharmaceutically acceptable salt thereof, wherein the tumor-associated macrophages are inhibited or depleted.

In another aspect, a method of targeting tumor-associated macrophages (e.g., pro-tumor M2-biased TAMs) in a host animal is provided. The method comprises the step of administering to the host animal a therapeutically effective amount of Conjugate 5, or a pharmaceutically acceptable salt thereof, wherein the tumor-associated macrophages are targeted.

In yet another illustrative aspect, a method for treating a cancer in a host animal wherein tumor-associated macrophages are in the cancer is provided. The method comprises the steps of administering to the host animal a therapeutically effective amount Conjugate 5, or a pharmaceutically acceptable salt thereof, and treating the cancer having the tumor-associated macrophages (e.g., pro-tumor M2-biased TAMs). In another embodiment Conjugate 5 includes a folate that binds the folate receptor-α and/or the folate receptor-β.

The phrase “wherein tumor-associated macrophages are in the cancer” used herein generally refers to the tumor associated macrophages (e.g., pro-tumor M2-biased TAMs) that exist in the microenvironment of a cancer (e.g., a tumor), or, for example, are found in cancerous tissue (e.g., tumor tissue).

The methods described herein are used to treat a “host animal” with cancer in need of such treatment. In one embodiment, the methods described herein can be used for both human clinical medicine and veterinary applications. Thus, a “host animal” can be administered the conjugate or folate-imaging agent conjugates described herein (described below), and the host animal can be human (e.g., a human patient) or, in the case of veterinary applications, can be a laboratory, agricultural, domestic, or wild animal. In one aspect, the host animal can be a human, a laboratory animal such as a rodent (e.g., mice, rats, hamsters, etc.), a rabbit, a monkey, a chimpanzee, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In various embodiments, the cancer described herein can be a cancer cell population that is tumorigenic, including benign tumors and malignant tumors, or the cancer can be non-tumorigenic. In another embodiment, the cancer can arise spontaneously or by such processes as mutations present in the germline of the host animal or by somatic mutations, or the cancer can be chemically-, virally-, or radiation-induced. Cancers applicable to the invention described herein include, but are not limited to, a carcinoma, a sarcoma, a lymphoma, a melanoma, a mesothelioma, a nasopharyngeal carcinoma, a leukemia, an adenocarcinoma, and a myeloma.

In some aspects the cancer can be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, prostate cancer, leukemia, lymphoma, pleural mesothelioma, cancer of the bladder, Burkitt's lymphoma, cancer of the ureter, cancer of the kidney, neoplasms of the central nervous system, brain cancer, pituitary adenoma, or adenocarcinoma of the gastroesophageal junction.

In some aspects the cancers can be selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkins lymphoma, uveal melanoma, glioblastoma, renal carcinoma, leiomyosarcoma, and pigmented villonodular synovitis. Any cancer that has tumor-associated macrophages (e.g., pro-tumor M2-biased TAMs) can be treated in accordance with the invention.

It is to be understood that Conjugate 5 described herein is the compound having the formula

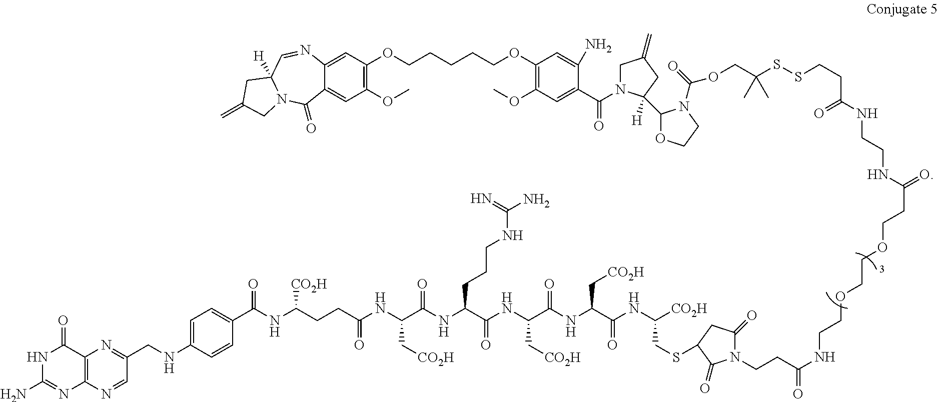

A pharmaceutically acceptable salt of Conjugate 5 can also be used.

Additional illustrative and non-limiting embodiments of the invention are described in the following enumerated clauses. All combinations of the following clauses are understood to be additional embodiments of the invention described herein.

1. A method for treating a folate receptor negative cancer comprising administering to the host animal a therapeutically effective amount of Conjugate 5, or a pharmaceutically acceptable salt thereof, wherein tumor-associated macrophages are inhibited or depleted.

2. A method for treating a folate receptor negative cancer comprising administering to the host animal a therapeutically effective amount of Conjugate 5, or a pharmaceutically acceptable salt thereof, to deplete tumor-associated macrophages.

3. A method for treating a folate receptor negative cancer comprising administering to the host animal a therapeutically effective amount of Conjugate 5, or a pharmaceutically acceptable salt thereof, and treating the folate receptor negative cancer having tumor-associated macrophages.

4. A method for treating a folate receptor negative cancer in a host animal comprising administering to the host animal a therapeutically effective amount of Conjugate 5, or a pharmaceutically acceptable salt thereof, to target tumor associated macrophages.

5. A method for treating a cancer comprising the steps of identifying the presence of tumor-associated macrophages in the cancer in a host animal, and administering to the host animal a therapeutically effective amount of Conjugate 5, or a pharmaceutically acceptable salt thereof.

6. A method for treating a cancer in a host animal, the method comprising the step of administering to the host animal a therapeutically effective amount of Conjugate 5, or a pharmaceutically acceptable salt thereof, to inhibit or deplete tumor-associated macrophages in the host animal.

7. A method for targeting tumor-associated macrophages in a host animal, the method comprising the step of administering to the host animal a therapeutically effective amount of Conjugate 5, or a pharmaceutically acceptable salt thereof, to target the tumor-associated macrophages.

8. A method for treating a cancer in a host animal where tumor-associated macrophages are in the cancer and/or form part of the tissue or tumor, the method comprising the steps of administering to the host animal a therapeutically effective amount of Conjugate 5, or a pharmaceutically acceptable salt thereof, and treating the cancer having the tumor-associated macrophages.

9. The method of any one of clauses 1 to 8 wherein tumor associated macrophages are in the cancer and the tumor-associated macrophages have the pro-tumor M2-biased CD163(+) phenotype.

10. The method of any one of clauses 1 to 8 wherein tumor-associated macrophages are in the cancer and the tumor-associated macrophages have the pro-tumor M2-biased CD163(+) and TGF-β(+) phenotype.

11. The method of any one of clauses 1 to 8 wherein tumor-associated macrophages are in the cancer and the tumor-associated macrophages have the pro-tumor M2-biased CD11b(+) phenotype.

12. The method of any one of clauses 1 to 8 wherein tumor-associated macrophages are in the cancer and the tumor-associated macrophages have the pro-tumor M2-biased CD163(+) and CD11b(+) phenotype.

13. The method of any one of clauses 1 to 8 wherein tumor-associated macrophages are in the cancer and the tumor-associated macrophages have the pro-tumor M2-biased F480(+) phenotype.

14. The method of any one of clauses 1 to 8 wherein tumor-associated macrophages are in the cancer and the tumor-associated macrophages have the pro-tumor M2-biased F480(+) and CD11b(+) phenotype.

15. The method of any one of clauses 1 to 8 wherein the tumor-associated macrophages are in the cancer and the tumor-associated macrophages are pro-tumor M2-biased and express one or more markers selected from the group consisting of CD163(+), IL10(+), Arg1(+), TGF-β(+), VEGF(+), CD206(+), CD11b(+), and F480(+) phenotype.

16. The method of any one of clauses 1 to 8 wherein tumor-associated macrophages are in the cancer and/or form part of the tissue or cancer and the tumor-associated macrophages are pro-tumor M2-biased and express one or more markers selected from the group consisting of CD163(+), IL10(+), Arg1(+), TGF-β(+), VEGF(+), CD206(+), CD11b(+), and F480(+) phenotype.

17. The method of any one of clauses 1 to 16 wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkin's lymphoma, uveal melanoma, glioblastoma, renal carcinoma, leiomyosarcoma, and pigmented villonodular synovitis.

18. The method of any one of clauses 1 to 17 wherein Conjugate 5, or a pharmaceutically acceptable salt thereof, is capable of depleting, or depletes the tumor-associated macrophages in the host animal.

19. The method of any one of clauses 1 to 18 wherein Conjugate 5, or a pharmaceutically acceptable salt thereof, is capable of inhibiting, or inhibits the activity of the tumor-associated macrophages in the host animal.

20. The method of any one of clauses 1 to 19 wherein Conjugate 5, or a pharmaceutically acceptable salt thereof, is administered to the host animal in a parenteral dosage form.

21. The method of clause 20 wherein the parenteral dosage form is selected from the group consisting of intradermal, subcutaneous, intramuscular, intraperitoneal, intravenous, and intrathecal dosage forms.

22. The method of any one of clauses 1 to 21 wherein the therapeutically effective amount is from about 0.05 μmol/kg to about 6.0 μmol/kg of host animal body weight.

23. The method of any one of clauses 1 to 22 wherein the therapeutically effective amount is from about 0.05 μmol/kg to about 4.0 μmol/kg of host animal body weight.

24. The method of any one of clauses 1 to 23 wherein the therapeutically effective amount is from about 0.05 μmol/kg to about 2.0 μmol/kg of host animal body weight.

25. The method of any one of clauses 1 to 24 wherein the therapeutically effective amount is from about 0.05 μmol/kg to about 1.0 μmol/kg of host animal body weight.

26. The method of any one of clauses 1 to 8 wherein tumor-associated macrophages are in the cancer and the tumor-associated macrophages have the pro-tumor M2-biased CD163(+), IL10(+), Arg1(+), TGF-β(+), VEGF(+), CD206(+), CD11b(+), and F480(+) phenotype.

The dosage of Conjugate 5, or a pharmaceutically acceptable salt thereof, can vary significantly depending on the condition of the host animal, the cancer being treated, the route of administration of Conjugate 5, or a pharmaceutically acceptable salt thereof, and tissue distribution, and the possibility of co-usage of other therapeutic treatments, such as radiation therapy or additional drugs in combination therapies. The therapeutically effective amount to be administered to a host animal is based on body surface area, mass, and physician assessment of condition of the host animal. Therapeutically effective amounts can range, for example, from about 0.05 mg/kg of host animal weight to about 30.0 mg/kg of host animal weight, or from about 0.01 mg/kg of host animal weight to about 5.0 mg/kg of host animal weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of host animal weight. The total therapeutically effective amount of Conjugate 5, or a pharmaceutically acceptable salt thereof, may be administered in single or divided doses and may, at the physician's discretion, fall outside of the typical range given herein.

In another embodiment, Conjugate 5, or a pharmaceutically acceptable salt thereof, can be administered in a therapeutically effective amount of from about 0.5 μg/m² to about 500 mg/m², from about 0.5 μg/m² to about 300 mg/m², or from about 100 μg/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount may be administered in single or divided doses and may, at the physician's discretion, fall outside of the typical range given herein. These amounts are based on m² of host animal surface area.

In another embodiment, Conjugate 5, or a pharmaceutically acceptable salt thereof, can be administered in a therapeutically effective amount of from about 0.05 μmol/kg to about 6.0 mol/kg, from about 0.05 μmol/kg to about 5.0 mol/kg, from about 0.05 μmol/kg to about 4.0 mol/kg, from about 0.05 μmol/kg to about 3.0 mol/kg, from about 0.05 μmol/kg to about 2.0 mol/kg, from about 0.05 μmol/kg to about 1.0 mol/kg, from about 0.05 μmol/kg to about 0.5 mol/kg, from about 0.05 μmol/kg to about 0.4 mol/kg, from about 0.05 μmol/kg to about 0.3 mol/kg, from about 0.05 μmol/kg to about 0.2 mol/kg, or from about 0.05 μmol/kg to about 0.1 mol/kg. The total therapeutically effective amount of Conjugate 5, or a pharmaceutically acceptable salt thereof, may be administered in single or divided doses and may, at the physician's discretion, fall outside of the typical range given herein. In each case, these amounts are “kg” of host animal weight.

Any effective regimen for administering Conjugate 5, or a pharmaceutically acceptable salt thereof, can be used. For example, Conjugate 5, or a pharmaceutically acceptable salt thereof, can be administered as single doses, or it can be divided and administered as a multiple-dose daily regimen. Further, a staggered regimen, for example, one to three days per week can be used as an alternative to daily treatment, and such an intermittent or staggered daily regimen is considered to be equivalent to every day treatment and within the scope of this disclosure. In one embodiment, the host animal is treated with multiple injections of Conjugate 5, or a pharmaceutically acceptable salt thereof. In one embodiment, the host animal, for example, may be injected multiple times with Conjugate 5, or a pharmaceutically acceptable salt thereof, for example, at 12-72 hour intervals or at 48-72 hour intervals. Additional injections of Conjugate 5, or a pharmaceutically acceptable salt thereof, can be administered to the host animal at intervals of days or months after the initial injections, and the additional injections prevent recurrence of disease.

In another embodiment Conjugate 5, or a pharmaceutically acceptable salt thereof, can be administered to the host animal, for example, for at least one hour, at least four hours, at least six hours, at least eight hours, at least ten hours, at least twelve hours, or at least twenty-four hours, or can be administered daily or weekly, such as once a day, two times a day, three times a day, every day, every other day, two times weekly, three times weekly, or any other suitable regimen may be used.

In one embodiment an imaging agent linked to a folate can be used to determine folate receptor status, and/or whether the cancer expresses folate receptors, and/or to identify the presence of TAMs associated with cancers. Exemplary folate-linked imaging agents are described in U.S. Pat. Nos. 7,128,893 and 9,731,035, incorporated herein by reference.

As used herein, the term “tumor associated macrophages” (TAMs) generally refers to macrophages that exist in the microenvironment of a cancer, for example, a tumor and have one or more markers consistent with TAMs.

As used herein, the term “inhibiting tumor associated macrophages” generally refers to reducing the activity or eliminating the activity of TAMs, such as by reducing or eliminating the ability of TAMs to stimulate angiogenesis in tumor tissue.

As used herein, the term “depleting tumor associated macrophages” generally refers to reducing the number of TAMs, eliminating TAMs, or repolarizing TAMs, including causing TAMs to shift from an M2 to an M1 phenotype.

As used herein, the term “pro-tumor” with reference to TAMs generally refers to TAMs that enhance tumorgenesis, such as, for example, by inhibiting B and/or T cell activation, inhibiting tumor-associated antigen presentation, inhibiting cytotoxic granule release, and/or increasing angiogenesis.

As used herein, the term “M2-biased” generally refers to TAMs that are pro-tumor TAMs which may include TAMS that are M1 and that may shift from an M1 to M2 phenotype.

As used herein, the term “composition” generally refers to any product comprising more than one ingredient. It is to be understood that the compositions described herein may be prepared from isolated Conjugate 5 described herein or from salts, solutions, hydrates, solvates, and other forms of Conjugate 5 described herein. It is appreciated that certain functional groups, such as the hydroxy, amino, and like groups may form complexes with water and/or various solvents, in the various physical forms of Conjugate 5. It is also to be understood that the compositions may be prepared from various amorphous, non-amorphous, partially crystalline, crystalline, and/or other morphological forms of Conjugate 5, or a pharmaceutically acceptable salt thereof, described herein. It is also to be understood that the compositions may be prepared from various hydrates and/or solvates of Conjugate 5, or a pharmaceutically acceptable salt thereof, described herein. Accordingly, such pharmaceutical compositions that recite Conjugate 5, or a pharmaceutically acceptable salt thereof, described herein are to be understood to include each of, or any combination of, the various morphological forms and/or solvate or hydrate forms of Conjugate 5, or a pharmaceutically acceptable salt thereof, described herein.

As used herein, the term “therapeutically effective amount” refers to an amount of the conjugate, or pharmaceutically acceptable salt thereof, that elicits the biological or medicinal response in a subject (i.e. a tissue system, animal or human) that is being sought by a researcher, veterinarian, medical doctor or other clinician, which includes, but is not limited to, alleviation of the symptoms of the disease or disorder being treated. In one aspect, the therapeutically effective amount is that amount of an active which may treat or alleviate the disease or symptoms of the disease at a reasonable benefit/risk ratio applicable to any medical treatment. In another aspect, the therapeutically effective amount is that amount of an inactive prodrug of Conjugate 5, which when converted through normal metabolic processes to produce an amount of active Conjugate 5, or a pharmaceutically acceptable salt thereof, capable of eliciting the biological or medicinal response in a subject that is being sought.

It is also appreciated that the dose of Conjugate 5, or a pharmaceutically acceptable salt thereof, whether referring to monotherapy or combination therapy, is advantageously selected with reference to any toxicity, or other undesirable side effect, that might occur during administration of Conjugate 5, or a pharmaceutically acceptable salt thereof, described herein. Further, it is appreciated that the co-therapies described herein may allow for the administration of lower doses of Conjugate 5, or a pharmaceutically acceptable salt thereof, that show such toxicity, or other undesirable side effect, where those lower doses are below thresholds of toxicity or lower in the therapeutic window than would otherwise be administered in the absence of a cotherapy.

As used herein, “administering” includes all means of introducing Conjugate 5, or a pharmaceutically acceptable salt thereof, described herein to the host animal, including, but are not limited to, oral (po), intravenous (iv), intramuscular (im), subcutaneous (sc), transdermal, inhalation, buccal, ocular, sublingual, vaginal, rectal, and the like. The conjugates and compositions described herein may be administered in unit dosage forms and/or formulations containing conventional nontoxic pharmaceutically-acceptable carriers, adjuvants, and/or vehicles.

As used herein “pharmaceutical composition” or “composition” refers to a mixture of Conjugate 5 described herein, or pharmaceutically acceptable salts, solvates, hydrates thereof, with other chemical components, such as pharmaceutically acceptable excipients. The purpose of a pharmaceutical composition is to facilitate administration of a conjugate to a host animal. Pharmaceutical compositions suitable for the delivery of Conjugate 5, or a pharmaceutically acceptable salt thereof, described herein and methods for their preparation will be readily apparent to those skilled in the art. Such compositions and methods for their preparation may be found, for example, in ‘Remington's Pharmaceutical Sciences’, 19th Edition (Mack Publishing Company, 1995).

A “pharmaceutically acceptable excipient” refers to an inert substance added to a pharmaceutical composition to further facilitate administration of Conjugate 5, or a pharmaceutically acceptable salt thereof, such as a diluent or a carrier.

Conjugate 5, or a pharmaceutically acceptable salt thereof, described herein can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas; including associated cancers resistant to treatment modalities, such as therapeutic agents. Resistant cancers include, but are not limited to paclitaxel resistant cancers, and platinum resistant cancers, such as those cancers resistant to platinum drugs, such as cisplatin, carboplatin, oxaplatin, nedaplatin, and the like. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, stomach and lung cancers. In some embodiments, the cancer cell population produces a cancer, such as lung cancer, bone cancer, pancreatic cancer, skin cancer, cancer of the head or neck, cutaneous or intraocular melanoma, ovarian cancer, rectal cancer, cancer of the anal region, stomach cancer, colon cancer, breast cancer, triple negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma and pituitary adenoma.

The Conjugate 5, or a pharmaceutically acceptable salt thereof, or compositions described herein may be administered orally. Oral administration may involve swallowing, so that the Conjugate 5, or a pharmaceutically acceptable salt thereof, or composition enters the gastrointestinal tract, or buccal or sublingual administration may be employed by which the Conjugate 5, or a pharmaceutically acceptable salt thereof, or composition enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The Conjugate 5, or a pharmaceutically acceptable salt thereof, described herein may also be used in fast-dissolving, fast disintegrating dosage forms such as those described in Expert Opinion in Therapeutic Patents, 11 (6), 981-986, by Liang and Chen (2001). For tablet dosage forms, depending on dose, the Conjugate 5, or a pharmaceutically acceptable salt thereof, may make up from 1 weight % to 80 weight % of the dosage form, more typically from 5 weight % to 60 weight % of the dosage form. In addition to the Conjugate 5, or a pharmaceutically acceptable salt thereof, and compositions described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise Conjugate 5, or a pharmaceutically acceptable salt thereof, as described herein, a film-forming polymer, a binder, a solvent, a humectant, a plasticizer, a stabilizer or emulsifier, a viscosity-modifying agent and a solvent. Some components of the formulation may perform more than one function.

Solid formulations for oral administration may be formulated to be immediate and/or modified release. Modified release formulations include delayed-, sustained-, pulsed-, controlled-, targeted and programmed release.

Thus Conjugate 5, or a pharmaceutically acceptable salt thereof, described herein can be formulated as a solid, semi-solid, or thixotropic liquid for administration as an implanted depot providing modified release of the active compound. Examples of such formulations include drug-coated stents and poly(lactic-coglycolic)acid (PGLA) microspheres. Other suitable modified release formulations for the purposes of the disclosure are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The Conjugate 5, or a pharmaceutically acceptable salt thereof, described herein can also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous.

Suitable devices for parenteral administration include needle (including microneedle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of Conjugate 5, or a pharmaceutically acceptable salt thereof, described herein used in the preparation of parenteral solutions may be increased by the use of appropriate formulation techniques, such as the incorporation of solubility-enhancing agents.

The Conjugate 5, or a pharmaceutically acceptable salt thereof, described herein can also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm Sci, 88 (10), 955-958 by Finnin and Morgan (October 1999). Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release. The Conjugate 5, or a pharmaceutically acceptable salt thereof, described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. The pressurized container, pump, spray, atomizer, or nebulizer contains a solution or suspension of the Conjugate 5, or a pharmaceutically acceptable salt thereof, of the present disclosure comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the Conjugate 5, or a pharmaceutically acceptable salt thereof, may be micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules (made, for example, from gelatin or hydroxypropylmethylcellulose), blisters and cartridges for use in an inhaler or insufflator may be formulated to contain a powder mix of the Conjugate 5, or a pharmaceutically acceptable salt thereof, described herein, a suitable powder base such as lactose or starch and a performance modifier such as Iso-leucine, mannitol, or magnesium stearate.

The lactose may be anhydrous or in the form of the monohydrate, preferably the latter. Other suitable excipients include dextran, glucose, maltose, sorbitol, xylitol, fructose, sucrose and trehalose. A typical formulation may comprise Conjugate 5, or a pharmaceutically acceptable salt thereof, of the present disclosure, propylene glycol, sterile water, ethanol and sodium chloride. Alternative solvents which may be used instead of propylene glycol include glycerol and polyethylene glycol.

The Conjugate 5, or a pharmaceutically acceptable salt thereof, described here can be combined with soluble macromolecular entities, such as cyclodextrin and suitable derivatives thereof or polyethylene glycol containing polymers, in order to improve their solubility, dissolution rate, taste-masking, bioavailability and/or stability for use in any of the aforementioned modes of administration.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

It is appreciated that Conjugate 5, or a pharmaceutically acceptable salt thereof, described herein may exist in unsolvated forms as well as solvated forms, including hydrated forms. In general, the solvated forms are equivalent to unsolvated forms and are encompassed within the scope of the present invention.

In another embodiment, compositions and/or dosage forms for administration of the Conjugate 5, or a pharmaceutically acceptable salt thereof, are prepared from the Conjugate 5, or a pharmaceutically acceptable salt thereof, with purity of at least about 90%, or about 95%, or about 96%, or about 97%, or about 98%, or about 99%, or about 99.5%. In another embodiment, compositions and or dosage forms for administration of the Conjugate 5, or a pharmaceutically acceptable salt thereof, are prepared from the Conjugate 5, or a pharmaceutically acceptable salt thereof, with a purity of at least 90%, or at least 95%, or at least 96%, or at least 97%, or at least 98%, or at least 99%, or at least 99.5%.

### EXAMPLES

### Chemical Examples

It is to be understood that the conjugates and compounds described herein were prepared according to the processes described herein and/or conventional processes. Illustratively, the stereocenters of the conjugates described herein may be substantially pure (S), the substantially pure (R), or any mixture of (S) and (R) at any asymmetric carbon atom, and each may be used in the processes described herein. Similarly, the processes described in these illustrative examples may be adapted to prepare other conjuagtes described herein by carrying out variations of the processes described herein with routine selection of alternative starting materials and reagents.

### Example 1: Preparation of Compound 6

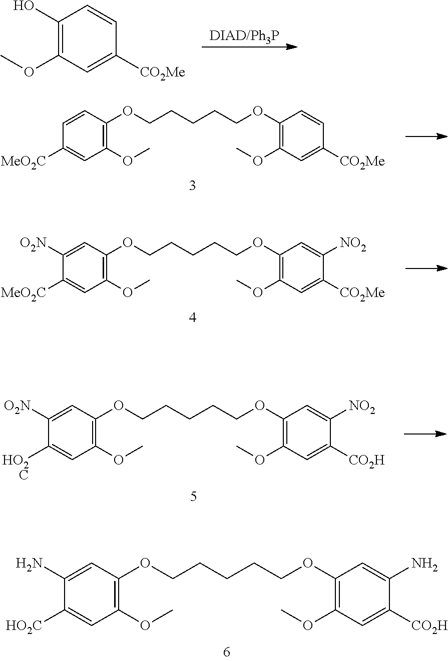 [SMILES: COC(=O)c1ccc(O)c(OC)c1 ; COC(=O)c1ccc(OCCCCCOc2ccc(C(=O)OC)cc2OC)c(OC)c1 ; COC(=O)c1cc(OC)c(OCCCCCOc2cc([N+](=O)[O-])c(C(=O)OC)cc2OC)cc1[N+](=O)[O-] ; COc1cc(C(=O)O)c([N+](=O)[O-])cc1OCCCCCOc1cc([N+](=O)[O-])c(C(=O)O)cc1OC ; COc1cc(C(=O)O)c(N)cc1OCCCCCOc1cc(N)c(C(=O)O)cc1OC]

### Step 1: Preparation of Compound 3

Methyl vanillate (2.18 g, 11.98 mmol) and Ph₃P (4.71 g, 17.97 mmol) in THF (20 mL) was cooled to 0° C. and to which was added DIAD (2.59 mL, 13.18 mmol) dropwise. The reaction was stirred at 0° C. for 1 hr. 1,5-petanediol (0.6 mL, 5.75 mmol) in THF (20 mL) was added over 30 min. The reaction was stirred overnight and precipitate formed and was collected with filtration. The filtrate was concentrated to form more solid. The solid was combined and triturated with MeOH (5 mL) to give clean product Compound 3 1.74 g in yield of 70%. ¹H NMR (CDCl₃, δ in ppm): 7.66 (m 2H), 7.62 (m, 2H), 6.87 (m, 2H), 4.10 (m, 4H), 3.89 (m, 12H), 1.95 (m, 4H), 1.69 (m, 2H). ¹³C NMR: 166.88, 152.50, 148.86, 132.12, 132.04, 131.88, 128.52, 128.42, 123.50, 122.55, 112.35, 111.46, 68.67, 56.03, 51.93, 28.73, 22.52, 21.92.

### Step 2: Preparation of Compound 4

Compound 3 (201.2 mg, 0.465 mmol) in Ac₂O (1.2 mL) was cooled to 0° C. and then Cu(NO₃)₂. 3H₂O (280.3 mg, 1.16 mmol) was added slowly and after 1 hr, the ice-bath was removed. The reaction was stirred at r.t. for 4 hours. The reaction was poured into ice water and stirred for 1 hour till yellow precipitate formed and was collected with filtration. The solid was washed with more cold water (2 mL, 3×) and air-dried. 198.4 mg of Compound 4 was obtained in yield of 82%. LCMS: [M+NH₄]⁺ m/z=540.

### Step 3: Preparation of Compound 5

Compound 4 (198.4 mg) was dissolved in THF (2 mL) and treated with aq. NaOH (2 mL, 1 M) and heated to 40° C. for 3 hours. The solvent was removed in vacuo. The aqueous phase was acidified to pH 1 with concentrated HCl to form precipitate, which was collected by filtration and was washed with H₂O (1 mL, 3×). The solid was air-dried to give the acid 187.7 mg of Compound 5 in quantitative yield. LCMS: [M+NH₄]⁺ m/z=512.

### Step 4: Preparation of Compound 6

Acid Compound 5 was dissolved in 0.5 M aq. NaOH (6 mL) and hydrogenation was carried out with Pd/C (10%, 4.82 mg) under H₂ (45 PSI) in the hydrogenation parr reactor. The reaction was shook for 5 hours and the filtered through a pad of celite and the filtrate was adjusted to pH 2-3 with concentrated HCl while stirring. The formed precipitate was isolated by filtration and washed with H₂O (1 mL, 3×). The solid was dried in a desiccator with the presence of P₂O₅ under high vacuum overnight. Compound 6 was obtained 34.2 mg as a brown solid in the yield of 81%. LCMS: [M−H]⁻ m/z=433.

### Example 2: Preparation of Compound 8

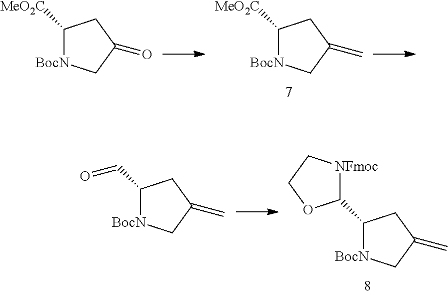

### Step 1: Preparation of Compound 7

(S)-1-tert-butyl 2-methyl 4-oxopyrrolidine-1,2-dicarboxylate was converted to Compound 7 by Wittig reaction: Ph₃PCH₃Br (917.8 mg, 2.57 mmol) in THF (30 mL) was treated with KO′Bu (1 M in THF, 2.57 μL, 2.57 mmol) at 0° C. by dropwise addition. The reaction was kept at room temperature for 2 hours. Into the stirred solution was added the ketone (250 mg, 1.028 mmol) in THF 20 mL) at 0-10° C. The reaction was then stirred at room temperature for overnight. The reaction was quenched with H₂O/EtOAc (1:1, 40 mL) after most of the THF was removed in vacuo. The aq. phase was extracted with EtOAc (20 mL, 3×) and the organic phase was washed with H₂O, followed by brine, and dried over anhydrous Na₂SO₄ and concentrated. The residue was purified with CombiFlash in 0-50% EtOAc/p-ether to afford the Compound 7 77.2 mg, in yield of 31%. LCMS: [M-Boc+H]⁺ m/z=142.

### Step 2: Preparation of Aldehyde Intermediate

(S)-1-tert-butyl 2-methyl 4-methylenepyrrolidine-1,2-dicarboxylate (353.2 mg, 1.46 mmol) in DCM/toluene (1:3, 9.8 mL) was treated with Dibal (1 M in toluene, 2 eq, 2.92 mmol) dropwise at −78° C. under argon. The reaction was stirred at −78° C. for ca. 4 hours. Then the reaction was quenched with addition of 60 μL of MeOH at −78° C. followed by 5% HCl (0.5 mL) and EtOAc (18 mL). The cold bath was removed and the reaction was stirred for 30 minutes. The EtOAc layer was separated and washed with brine, dried over anhydrous Na₂SO₄ and concentrated to give the crude aldehyde intermediate.

### Step 3: Preparation of Compound 8

The crude aldehyde was redissolved in dry DCM (10 mL) and treated with ethanolamine (106 μL, 1.75 mmol) in the presence of anhydrous MgSO₄ (5 mmol, mg) at r.t. (room temperature) under Ar. The reaction was stirred for 1 hour. Then into this reaction mixture was added FmocCl (755.4 mg, 2.92 mmol) and TEA (611 μL, 4.38 mmol) and the reaction was stirred for overnight at r.t. under Ar. The reaction was purified with CombiFlash in 0-50% EtOAc/petroleum ether to provide Compound 8 334.2 mg, 46% for 3 steps. LCMS: [M+H]⁺ m/z=477. ¹H NMR (CD₃OD, δ in ppm): 7.81 (d, J=7.5 Hz, 2H), 7.60 (d, J=7 Hz, 2H), 7.40 (m, 2H), 7.32 (m, 2H), 4.96 (br, 2H), 4.60 (br, 1H), 4.23 (t, J=5.5 Hz, 1H), 3.97 (br, 2H), 3.73 (br, m, 3H), 2.50 (br, 2H), 1.47 (s, 1H), 1.39 (s, 9H).

### Example 3: Preparation of Compound 9

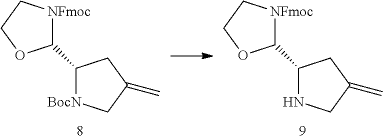

Compound 8 was deprotected in TFA/DCM (1:1) at r.t. for 30 min, the solvent was removed in vacuo.

### Example 4: Preparation of Compound 23

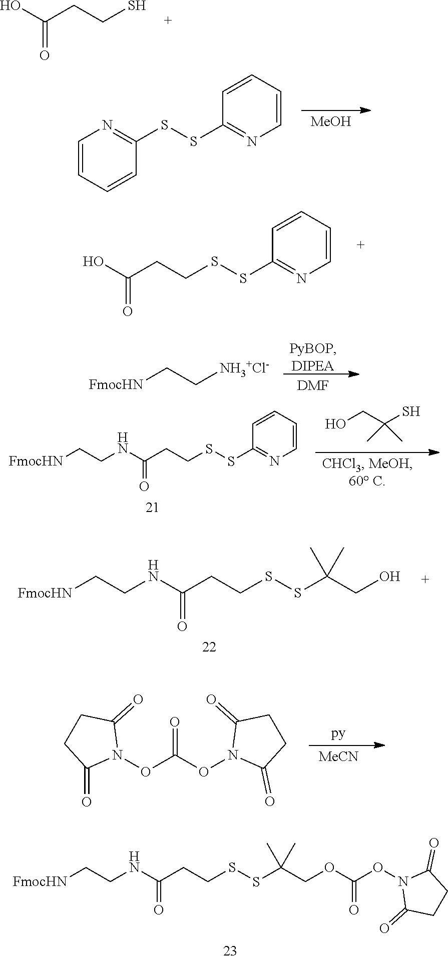

### Step 1: Preparation of 3-(2-Pyridyldithio)propionic Acid

2,2′-dipyridyl disulfide (8.70 g, 39.5 mmol) was dissolved in MeOH (150 mL) and purged with argon for 20 minutes. 3-Mercaptopropionic acid (2.10 g, 19.8 mmol) was dissolved in MeOH (35 mL) and purged under argon for 15 minutes. The 3-mercaptopropionic acid solution was added slowly to the 2,2′-dipyridyl disulfide solution using an addition funnel. The reaction was monitored by LC/MS, and after complete consumption of 3-mercaptopropionic acid, the reaction mixture was concentrated and loaded onto a 120 g C18 column. The purification was carried out with MeCN/H₂O (0-100%). The fractions were analyzed on LC/MS, and fractions containing the desired product were combined and evaporated under reduced pressure. An oil phase was observed on the bottom of the flask during concentration. This oily residue was separated from the aqueous phase and dried under high vacuum to yield the desired product as colorless solid (2.4 g). The aqueous phase was extracted with EtOAc in order to separate additional product. The organic extract was washed with brine, dried over Na₂SO₄, and concentrated in vacuo to yield the desired product (0.5 g). 3-(2-Pyridyldithio)propionic acid was isolated as a white solid (2.9 g, 68%); LC/MS (ESI-QMS): m/z=216.25 (M+H), ¹H NMR (CD₃OD): 8.39 (m, 1H), 7.84 (m, 1H), 7.79 (m, 1H), 7.21 (m, 1H), 4.87 (br, 1H), 3.03 (t, J=6.8 Hz, 2H), 2.70 (t, J=6.8 Hz, 2H). ¹³C NMR (CD₃OD): 173.53, 159.82, 148.97, 137.74, 120.99, 119.81, 33.50, 32.96.

### Step 2: Preparation of Compound 21

To a solution of N-Fmoc-ethylenediamine hydrochloride (500 mg, 1.57 mmol), 3-(2-Pyridyldithio)propionic acid (338 mg, 1.57 mmol), and ⁱPr₂NEt (839 uL, 4.71 mmol) in DMF (7.85 mL) was added PyBOP (950 mg, 1.57 mmol) in one portion. The reaction mixture was stirred for 5 minutes at room temperature and then concentrated under high vacuum. Water was added to the crude mixture (50 mL) and extracted with ethyl acetate (3×30 mL). The combined organic layers were dried over sodium sulfate, filtered, and evaporated to dryness to yield a pale yellow oil. The product was further purified via silica chromatography (0-80% EtOAc/pet. ether). The product was isolated as a white solid with 86% purity according to HPLC (633 mg, 84.1%): LC/MS (ESI-QMS): m/z=480.56 (M+H), ¹H NMR (500 MHz, CDCl₃) δ 8.44 (d, J=4.9, 1H), 7.75 (d, J=7.3, 2H), 7.59 (m, 3H), 7.40 (t, J=7.3, 2H), 7.30 (t, J=7.3, 2H), 7.09 (t, J=5.9, 1H), 6.98 (s, 1H), 4.56 (d, J=6.8, 2H), 4.17 (t, J=6.8, 1H), 3.43 (m, 2H), 3.40 (m, 2H), 3.08 (t, J=6.4, 2H), 2.60 (t, J=6.4, 2H).

### Step 3: Preparation of Compound 22

In a dry flask, Compound 21 (318 mg, 0.664 mmol, 1.0 equiv.) and 2-mercapto-2-methyl-propan-1-ol (92 mg, 0.863 mmol, 1.3 equiv.) were dissolved in CHCl₃:MeOH (1:3, 20 mL). The reaction mixture was stirred for 4 hours at 60° C. and monitored until completion by LC/MS. The solvent was removed under reduced pressure to yield an oily residue, followed addition of water and subsequent extractions with EtOAc (3×). The organic extracts were combined, dried over Na₂SO₄, filtered, and concentrated under reduced pressure. The product was further purified using silica gel chromatography (CH₂Cl₂/MeOH, 0-4%) to yield Compound 22 (285 mg, 90%): LC/MS (ESI-QMS): m/z=475.18 (M+H), ¹H NMR (500 MHz CDCl₃) δ 7.78 (d, J=7.3 Hz, 2H), 7.67 (d, J=7.3 Hz, 2H), 7.40 (dd, J=14.7, 7.9 Hz, 2H), 7.32 (dd, J=14.7, 7.9 Hz, 2H), 6.38 (s, 1H), 5.35 (s, 1H), 4.40 (d, J=6.9 Hz, 2H), 4.21 (dd, J=13.7, 6.8 Hz, 1H), 3.47 (s, 2H), 3.42-3.31 (m, 4H), 2.82 (t, J=6.9 Hz, 2H), 2.58 (t, J=6.9 Hz, 2H), 1.25 (s, 6H).

### Step 4: Preparation of Compound 23

To a suspension of Compound 22 (0.552 mg, 1.16 mmol) in dry MeCN (12 mL) under argon was added N,N′-disuccinimidyl carbonate (0.358 g, 1.40 mmol) and pyridine (0.118 mL, 1.45 mmol) respectively. The reaction was allowed to stir for 15 hours at room temperature in which the reaction turned into clear solution. LC/MS analysis confirmed that the reaction went to completion. The reaction mixture was concentrated and purified via silica chromatography (0-5% CH₂Cl₂/MeOH) to yield Compound 23 (0.68 g, 95%): LC/MS (ESI-QMS): m/z=616.24 (M+H), ¹H NMR (500 MHz, CD3OD) δ 7.79 (d, J1=7.5 Hz, 2H), 7.64 (d, J1=7.0 Hz, 2H), 7.38 (dd, J1=8.0 Hz, J2=7.5 Hz, 2H), 7.30 (dd, J1=7.0 Hz, J2=7.5 Hz, 2H), 4.33 (d, J1=7.0 Hz, 2H), 4.28 (s, 2H), 4.19 (t, J1=7.0 Hz, J2=6.5 Hz, 1H), 3.20-3.30 (m, 4H), 2.91 (t, J1=7.0 Hz, J2=7.0 Hz, 2H), 2.80 (s, 4H), 2.56 (t, J1=7.5 Hz, J2=7.5 Hz, 2H), 1.31 (s, 6H); ¹³C NMR (125 MHz, CD3OD) δ 172.41, 169.81 (2C), 157.60, 151.59, 143.92 (2C), 141.19 (2C), 127.37 (2C), 126.74 (2C), 124.79 (2C), 119.53 (2C), 75.90, 66.40, 48.39 (2C), 39.83, 39.05, 35.58, 35.12, 24.98 (2C), 23.05 (2C).

### Example 5: Preparation of Compound 26

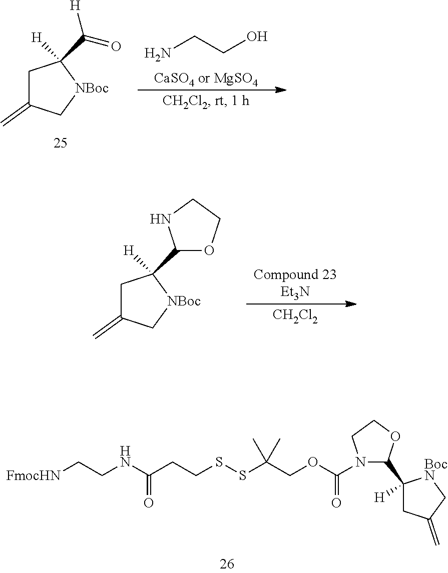

To a solution of the N-Boc-4-methylene-L-prolinal (44.36 mg, 0.2099 mmol) in dry CH₂Cl₂ (1 mL) was added anhydrous CaSO₄ (22 mg, 0.16 mmol) and ethanolamine (10.56 μL, 0.1750 mmol) respectively. The reaction was allowed to stir for 1 hour at room temperature. In another flask, Compound 23 (108 mg, 0.180 mmol) was dissolved in dry CH₂Cl₂ (1 mL). The previous pyrrolidine solution was filtered and slowly added to the Compound 23 solution. Et₃N (0.037 mL, 0.26 mmol) was added to the reaction mixture, and the resulting mixture was monitored via LC/MS. After stirring for 2 hours, the reaction mixture was diluted with CH₂Cl₂, washed with sat. NH₄Cl_{(aq)}, dried over Na₂SO₄, and concentrated in vacuo. The residue was further purified silica chromatography (0-10% CH₂Cl₂/MeOH) to yield pure Compound 26 (83 mg, 63%): LC/MS (ESI-QMS): m/z=755.38 (M+H), ¹H NMR (500 MHz, CD₃OD) δ 7.79 (d, J1=8.0 Hz, 2H), 7.64 (d, J1=7.5 Hz, 2H), 7.38 (dd, J1=7.5 Hz, J2=7.5 Hz, 2H), 7.30 (dd, J1=7.5 Hz, J2=7.5 Hz, 2H), 5.13-5.20 (m*, 1H), 4.88-5.05 (m*, 2H), 4.36-4.60 (m*, 1H), 4.33 (d, J1=7.0 Hz, 2H), 4.20 (t, J1=7.0 Hz, J2=7.0 Hz, 1H), 3.98-4.10 (m*, 3H), 3.72-3.94 (m*, 4H), 3.36-3.50 (m*, 1H), 3.18-3.30 (m*, 4H), 2.91 (t, J1=7.5 Hz, J2=7.0 Hz, 2H), 2.70-2.40 (m*, 2H), 2.54 (t, J1=7.0 Hz, J2=7.0 Hz, 2H), 1.40-1.50 (m*, 9H), 1.26-1.38 (m*, 6H). *Due to diasteromeric and/or rotameric nature of the compound

### Example 16: Preparation of Compound 29

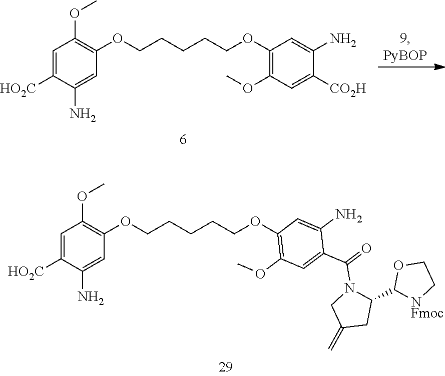

Compound 6 (42.0 mg, 0.097 mmol), Compound 9 (0.053 mmol), and PyBOP (29.0 mg, 0.056 mmol) were dissolved in DMF/DCM (0.5 mL/0.5 mL) and treated with DIPEA (74 μL, 0.43 mmol) at r.t. under Ar. The reaction was completed within 1 hr, then loaded onto CombiFlash column in 0-20% MeOH/DCM to afford the pure product Compound 29 (25.5 mg, 60%). LCMS: [M+H]⁺ m/z=793.

### Example 6: Preparation of Compound 32

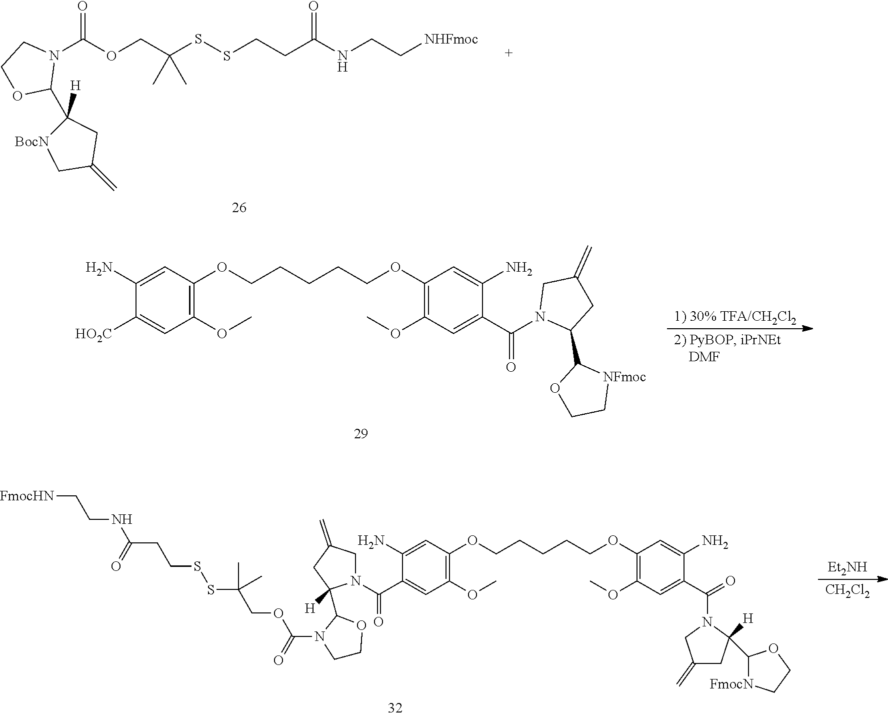

### Step 1: Preparation of Compound 32

In a flask, Compound 26 (95.0 mg, 0.126 mmol) was dissolved in 30% TFA/CH₂Cl₂ (10 mL) at 0° C. The reaction mixture was allowed to warm to room temperature and stirred for 1 hour. Upon complete removal of the Boc protecting group, the solvent was removed under reduced pressure, and the crude residue was left under high vacuum for 3 hours. In a dry flask, the crude TFA salt and Compound 29 (100 mg, 0.126 mmol) were dissolved in dry DMF (2.5 mL) under argon. To the reaction mixture was added PyBOP (131 mg, 0.252 mmol) and ⁱPr₂NEt (67 μl, 0.378 mmol) subsequently. After 3 hours, the reaction was quenched by the addition of sat. NH₄Cl_{(aq)} and extracted with EtOAc (3×). The combined organic layers were dried over Na₂SO₄, filtered, and concentrated under reduced pressure. The product was purified using silica gel chromatography (0-8% MeOH/CH₂Cl₂) to yield Compound 32 (153 mg, 84.9%): LC/MS (ESI-QMS): m/z=1429.78 (M+H), ¹H NMR (500 MHz CDCl₃) δ Pivotal signals: δ 7.75-7.66 (m, 4H), 7.58-7.47 (m, 4H), 7.75-7.66 (m, 4H), 7.39-7.31 (m, 4H), 7.29-7.22 (m, 4H), 7.02-6.51 (m, 4H), 5.31-5.14 (m, 1H), 5.04-4.74 (m, 5H), 1.28-1.12 (m, 6H).

### Example 21: Preparation of Conjugate 5

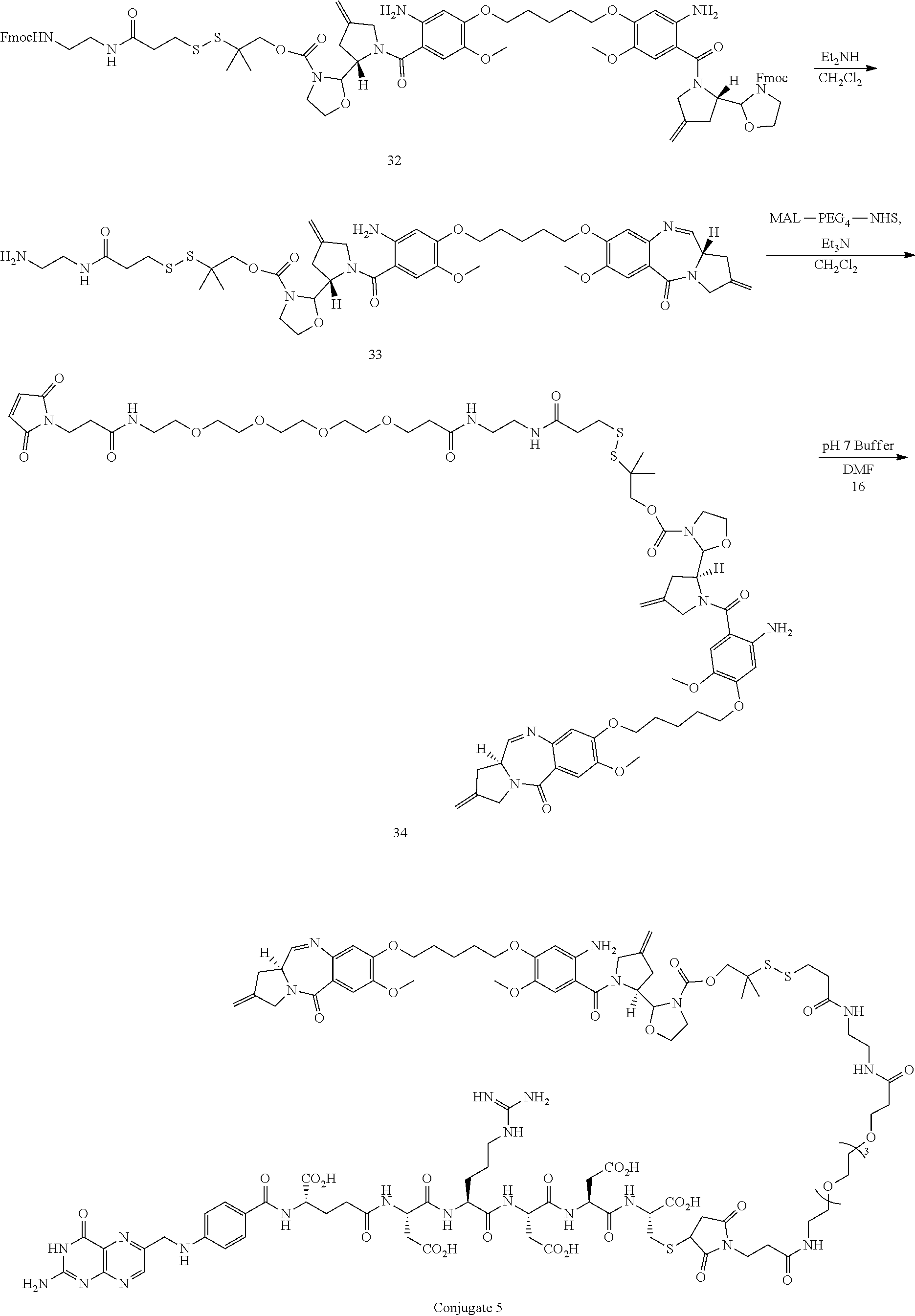

Compound 32 (23 mg, 0.016 mmol) and diethylamine (0.25 mL, 2.4 mmol) were dissolved in CH₂Cl₂ (0.6 mL), and the reaction mixture was stirred at room temperature under argon for 3 hours. The reaction was monitored via LC/MS and after complete consumption of Compound 32, the solvent was removed under reduced pressure. The resulting residue was co-evaporated with CH₂Cl₂ twice and dried under high vacuum for 15 minutes. The resulting residue was dissolved in CH₂Cl₂ (0.5 mL), and Mal-PEG4-NHS ester (10.9 mg, 0.021 mmol) and Et₃N (3.0 μL, 0.021 mmol) were added. The reaction was stirred at room temperature under argon and monitored via LC/MS for production of Compound 34 (m/z=1323 and 662). After 1 h, the reaction mixture was evaporated, and the resulting residue was dissolved in DMF (2 mL). The solution was purged with argon. Compound 16 (22 mg, 0.021 mmol), which was prepared according to the methods disclosed in PCT/US2011/037134 (WO2011146707), incorporated herein by reference for the preparation of Compound 16, was dissolved in pH 7 buffer (2 mL, 50 mM NH₄HCO₃), purged with argon, and added to the above Compound 34 solution. The reaction was stirred at room temperature while purging with argon. The reaction was monitored via LC/MS for the production of Conjugate 5 (m/z=791). After 2 hours, purification via preperative HPLC (10-100% MeCN/50 mM NH₄HCO₃ pH 7 buffer) yielded two sets of isomers: 1.9 mg of 1^{st} set of isomers with a shorter retention time and 7.4 mg of 2^{nd} set of isomers with a longer retention time. The desired product was obtained in a yield of 24% over three steps: LC/MS (ESI-QMS): m/z=791.25 (M+3H), Major Product: ¹H NMR (DMSO-D6, selected data): 8.61 (s, 1H), 7.72 (d, NH), 7.55 (d, J=8.8 Hz, 2H), 7.30 (s, NH), 7.15 (s, ArH), 7.01 (s, ArH), 6.81 (s, NH), 6.60 (d, J=8.8 Hz, 2H+1H overlapped), 6.54 (s, ArH), 6.34 (s, N═CH), 6.32 (s, ArH), 5.11+5.06 (m, 2H), 4.96+4.92+4.85 (m, 3H), 3.66+3.62 (s+s, 3H), 3.61 (s, 3H), 3.55 (t, 3H), 3.35 (t, 3H), 1.21 (s, br, 6H). Minor Product: ¹H NMR (DMSO-D6, selected data): 8.61 (s, 1H), 7.72 (d, NH), 7.55 (d, J=8.8 Hz, 2H), 7.29 (s, NH), 7.15 (s, ArH), 7.01 (s, ArH), 6.80 (s, NH), 6.60 (d, J=8.8 Hz, 2H+1H overlapped), 6.53 (s, ArH), 6.32 (s, N═CH), 6.31 (s, ArH), 5.11+5.06 (m, 2H), 4.94-4.85 (m, 3H), 3.66+3.62 (s+s, 3H), 3.61 (s, 3H), 3.55 (t, 3H), 3.35 (t, 3H), 1.20 (s, br, 6H).

### BIOLOGICAL EXAMPLES

### General.

The following abbreviations are used herein: partial response (PR); complete response (CR), once weekly (SIW), biweekly (M/F) (BIW), three times per week (M/W/F) (TIW). A PR is observed where tumor volume, as defined herein, decreases from a previous high during the observation period, though regrowth may occur. A CR is observed where tumor volume, as defined herein, decreases to zero during the observation period, though regrowth may occur. A cure is observed where tumor volume, as defined herein, decreases to zero, and does not regrow during the observation period.

### Method 1. Inhibition of Cellular DNA Synthesis.

The conjugates described herein were evaluated using an in vitro cytotoxicity assay that predicted the ability of the drug to inhibit the growth of the corresponding targeted cells, such as, but not limited to the following

Cell Line

| | |
|---|---|
| KB | Human cervical carcinoma |
| NCl/ADR-RES-Cl₂ | Human ovarian carcinoma |
| IGROV1 | Human ovarian adenocarcinoma |
| MDA-MB-231 | Human breast adenocarcinoma (triple negative) |
| A549 | Human lung carcinoma |
| H23 | Human lung adenocarcinoma |
| HepG2 | Human hepatocellular carcinoma |
| AN3CA | Human endometrial adenocarcinoma |
| 4T1p | Mouse breast carcinoma |
| 4T1-C12 | 4T1p transfected with human FRα |
| ID8-Cl15 | Ovarian carcinoma transfected with murine FR-α |

It is to be understood that the choice of cell type can be made on the basis of the susceptibility of those selected cells to the drug that forms the conjugate, and the relative expression of the cell surface receptor or target antigen. The test conjugates were conjugates of a cell surface receptor or target antigen binding compound and PBD prodrugs, poly-PBD prodrugs, and mixed PBDs, as described herein. The test cells were exposed to varying concentrations of the conjugates, and optionally also in the absence or presence of at least a 100-fold excess of the unconjugated cell surface receptor or target antigen binding compound for competition studies to assess activity as being specific to the cell surface receptor or target antigen.
Method 2: In Vitro Folate Receptor Specific Activity Assay of Folate conjugates.

KB cells were seeded in individual 24-well Falcon plates and allowed to form nearly confluent monolayers overnight in folate free Roswell Park Memorial Institute (FFRPMI)/Heat-Inactivated Fetal Calf Serum (HIFCS). Thirty minutes prior to the addition of folate-conjugate, spent medium was aspirated from all wells and replaced with either fresh FFRPMI or FFRPMI supplemented with 100 μM folic acid. Each well then received 1 mL of medium containing increasing concentrations of folate-conjugate (3 wells per sample). Cells were pulsed for 2 hours at 37° C., rinsed 4 times with 0.5 mL of medium and then chased in 1 mL of fresh medium up to 72 h. Spent medium was aspirated from all wells and replaced with fresh medium containing 5 μCi/mL of ³H-thymidine. Following a 2 hour incubation at 37° C., cells were washed 3 times with 0.5 mL of PBS and then treated with 0.5 mL of ice-cold 5% trichloroacetic acid per well. After 15 minutes, the trichloroacetic acid was aspirated and the cells solubilized by the addition of 0.5 mL of 0.25 N sodium hydroxide for 15 minutes at room temperature. Four hundred and fifty μL of each solubilized sample were transferred to scintillation vials containing 3 mL of Ecolume scintillation cocktail and counted in a liquid scintillation counter. Final results were expressed as the percentage of ³H-thymidine incorporation relative to untreated controls. For conjugates described herein, dose-dependent cytotoxicity was generally measurable, and in most cases, the IC₅₀ values (concentration of drug conjugate required to reduce ³H-thymidine incorporation into newly synthesized DNA by 50%) were in the picomolar to low nanomolar range.

### Example 1: Conjugate 5 in Vitro Activity

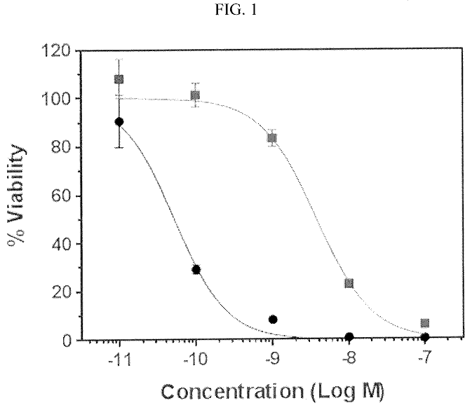

In FIG. 1, the percentage of ³H-thymidine incorporated into KB cells treated with Conjugate 5 (●) and with Conjugate 5 and excess folate (▪) is shown.

### Example 2: Relative Affinity Assay

FR-positive KB cells were seeded in 24-well Falcon plates and allowed to form adherent monolayers (>90% confluent) overnight in FFRPMI/HIFCS. Spent incubation medium was replaced with FFRPMI supplemented with 10% HIFCS and containing 100 nmol/L of [³H]FA in the absence and presence of increasing concentrations of unlabeled FA or the test conjugate. Cells were incubated for 1 hour at 37° C. and then rinsed thrice with 0.5 mL PBS. Five hundred microliters of 1% SDS in PBS were added to each well; after 5 min, cell lysates were collected, transferred to individual vials containing 5 mL of scintillation cocktail, and then counted for radioactivity.

Cells exposed to only the [³H]FA in FFRPMI (no competitor) were designated as negative controls, whereas cells exposed to the [³H]FA plus 1 mmol/L unlabeled FA served as positive controls. Disintegrations per minute (DPM) measured in the latter samples (representing nonspecific binding of label) were subtracted from the DPM values from all samples. Notably, relative affinities were defined as the inverse molar ratio of compound required to displace 50% of [³H]FA bound to FR on KB cells, and the relative affinity of FA for the FR was set to 1.

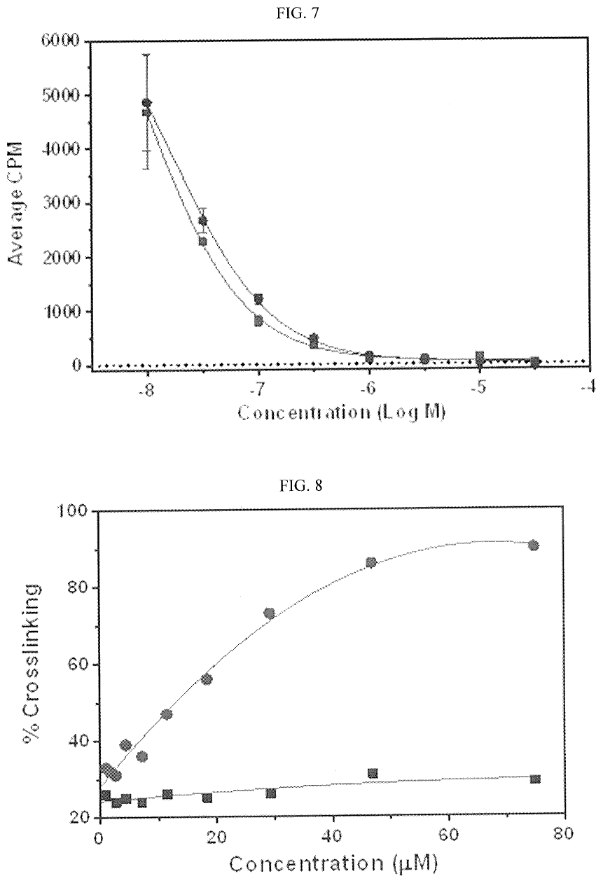

Results for Conjugate 5 are shown in FIG. 7. The results show that linkage of a large drug molecule does not radically alter the vitamin's intrinsic binding affinity to its receptor.

### Example 3: DNA Crosslinking Assay of Conjugate 5

Calf thymus DNA (CT-DNA) was combined with increasing concentrations of Conjugate 5 (1.1 to 75 μM) or Conjugate 5+/−DTT. These solutions were incubated at 37° C. for 2 hours. The solutions were then mixed with ethidium bromide and incubated for 2 hours at room temperature. Fluorescence (Ex: 535 nm, Em: 605 nm) from these samples was measured on the Fluoroskan II fluorimeter. Next, the samples were heated to 104° C. for 5 minutes, cooled on ice for 5 minutes, kept at RT for 15 minutes and fluorescence measured. % crosslinking of each sample was calculated using the fluorescence values from the positive and negative controls. Results are shown in FIG. 8.

### Example 4: Conjugate 5 In Vivo Activity Against Tumors

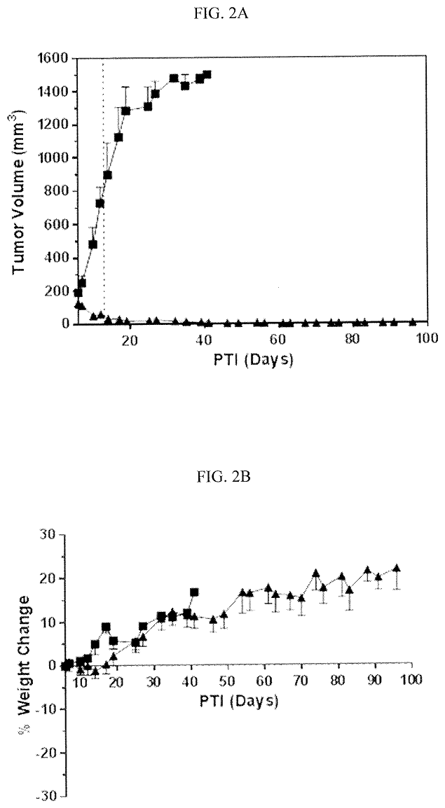

As shown in FIG. 2A, Conjugate 5 dosed at 0.5 μmol/kg SIW for two weeks (▴) decreased KB tumor size in test mice compared to untreated control (▪). Treatment with 0.5 μmol/kg of Conjugate 5, once a week for two weeks also produced maximal anti-tumor activity with 100% cures. Change in weight is shown in FIG. 2B for test mice dosed at 0.5 μmol/kg Conjugate 5 SIW for two weeks (▴) compared to untreated control (▪).

### Example 5: Conjugate 5 In Vivo Activity Against Paclitaxel Resistant Tumors

Mice were maintained and tumor volumes were measures according to Method 3.

KB-PR10 (paclitaxel resistant) tumor cells were inoculated subcutaneously at the right flank of each mouse. Mice were dosed through the lateral tail vein under sterile conditions in a volume of 200 mL of phosphate-buffered saline (PBS).

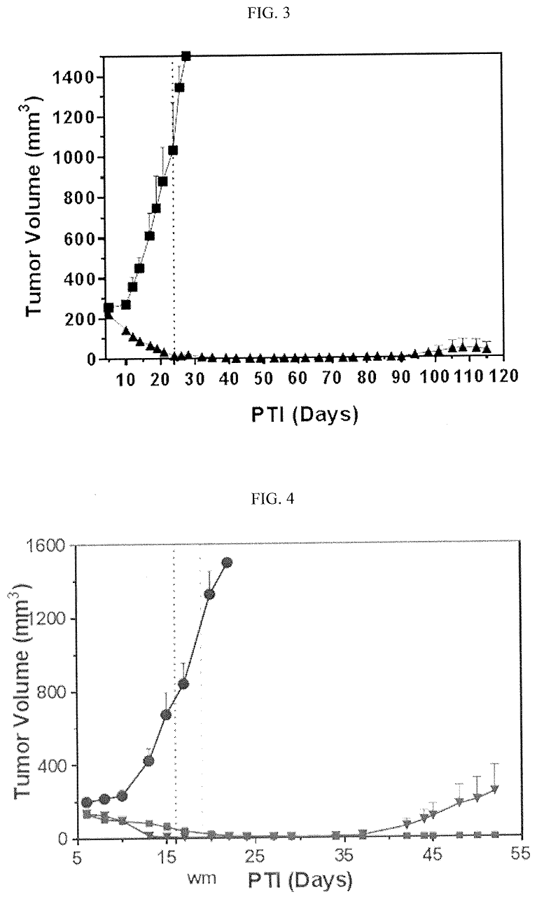

As shown in FIG. 3, Conjugate 5 dosed at 0.5 μmol/kg SIW for two weeks (▴) decreased paclitacel resistant KB tumor size in test mice compared to untreated control (▪).

### Example 6: Conjugate 5 In Vivo Activity Against Platinum Resistant Tumors

Mice were maintained and tumor volumes were measures according to Method 3.

KB-CR2000 (platin resistant) tumor cells were inoculated subcutaneously at the right flank of each mouse. Mice were dosed through the lateral tail vein under sterile conditions in a volume of 200 mL of phosphate-buffered saline (PBS).

As shown in FIG. 4, Conjugate 5 dosed at 0.5 μmol/kg SIW for two weeks (▪) and EC1456 (EC1456 is a conjugate folate linked to a tubulysin that is known in the art) dosed at 2.0 μmol/kg BIW for two weeks (▾) decreased paclitacel resistant KB tumor size in test mice compared to untreated control (●).

### Example 7: Conjugate 5 In Vivo Activity Against Triple Negative Breast Tumors

Mice were maintained and tumor volumes were measures according to Method 3.

Primary human TNBC model ST502 (2-4 mm in diameter) or primary human TNBC model ST738 (2-4 mm in diameter) were inoculated subcutaneously at the right flank of each mouse. Mice were randomized into experimental groups of 7 mice each and test articles were injected through the lateral tail vein under sterile conditions in a volume of 200 mL of phosphate-buffered saline (PBS).

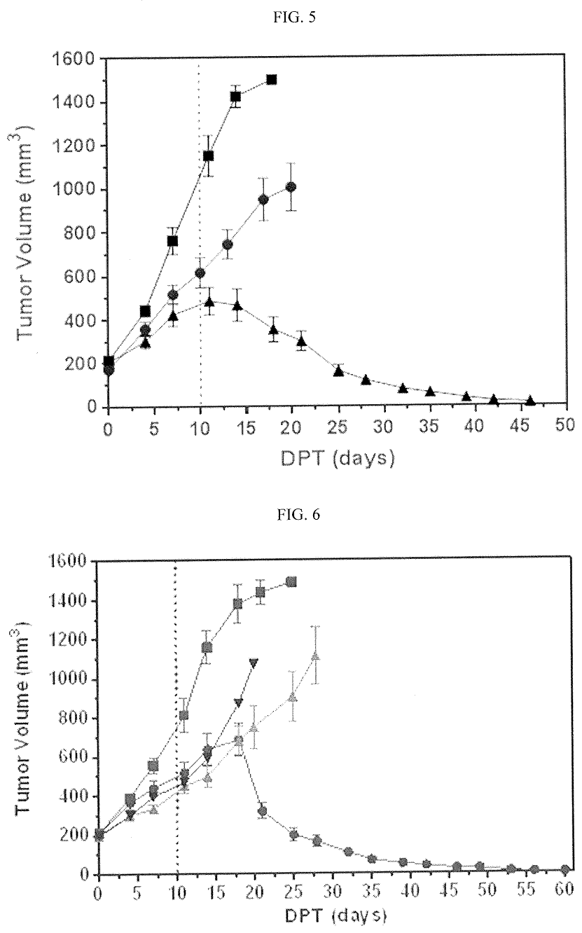

As shown in FIG. 5, Conjugate 5 dosed at 0.3 μmol/kg BIW for two weeks (▴) decreased TNBC PDX tumor size in test mice compared to untreated control (▪), whereas EC1456 dosed at 2.0 μmol/kg BIW for two weeks (●) did not decrease TNBC PDX tumor size.

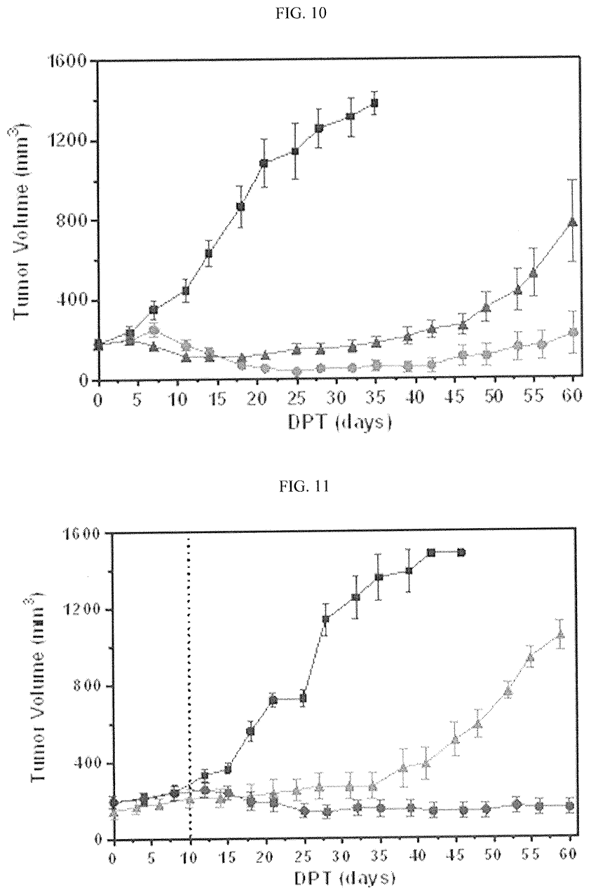

As shown in FIG. 10, Conjugate 5 dosed at 0.27 μmol/kg BIW for two weeks (▪) decreased TNBC PDX tumor size in test mice compared to untreated control (▪), whereas erubulin mesylate dosed at 1.0 μmol/kg SIW for two weeks (▴) did not decrease TNBC PDX tumor size.

### Example 8: Conjugate 5 In Vivo Activity Against Ovarian Tumors

Mice were maintained and tumor volumes were measures according to Method 3.

Primary human Ovarian model ST070 fragments (2-4 mm in diameter) were inoculated subcutaneously at the right flank of each mouse. Mice were randomized into experimental groups of 7 mice each and test articles were injected through the lateral tail vein under sterile conditions in a volume of 200 mL of phosphate-buffered saline (PBS).

As shown in FIG. 6, Conjugate 5 dosed at 0.5 μmol/kg SIW for two weeks (▪) decreased ovarian PDX tumor size in test mice compared to untreated control (▪), whereas EC1456 dosed at 4.0 μmol/kg SIW for two weeks (▴) and paclitaxel dosed at 15 mg/kg SIW for two weeks (▾) did not decrease ovarian PDX tumor size.

### Example 9: Conjugate 5 In Vivo Activity in KB Rat Tumor Model

Female Balb/c nu/nu rats were fed ad libitum with folate-deficient chow (Harlan diet # TD01013) for the duration of the experiment. KB-tumor cells were inoculated subcutaneously at the right flank of each rat. Rats were dosed through the lateral tail vein under sterile conditions in a volume of 200 mL of phosphate-buffered saline (PBS).

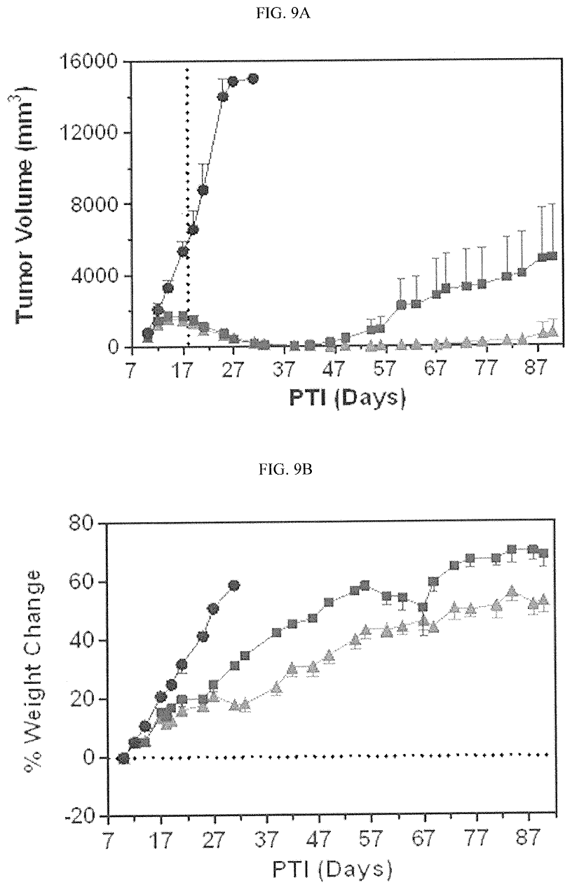

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm. Results for tumor volume are shown in FIG. 9A. Toxicity was measured as a function of animal weight gain or loss as shown in FIG. 9B.

### Example 10: Conjugate 5 In Vivo Activity Against Endopetrial Tumors

Female Balb/c nu/nu mice were fed ad libitum with folate-deficient chow (Harlan diet # TD01013) for the duration of the experiment. Primary human Endometrial model ST040 fragments (2-4 mm in diameter) were inoculated subcutaneously at the right flank of each mouse. Mice were randomized into experimental groups of 7 mice each and test articles were injected through the lateral tail vein under sterile conditions in a volume of 200 mL of phosphate-buffered saline (PBS). These studies were performed at South Texas Accelerated Research Therapeutics, 4383 Medical Drive, San Antonio, Tex. 78229.

Growth of each s.c. tumor was followed by measuring the tumor two times per week until a volume of 1200 mm³ was reached. Tumors were measured in two perpendicular directions using Vernier calipers, and their volumes were calculated as 0.5×L×W², where L=measurement of longest axis in mm and W=measurement of axis perpendicular to L in mm. FIG. 11 shows that treatment with paclitaxel at 15 mg/kg SIW for two weeks produced 0% partial response subjects, while Compound 5 dosed at 0.27 mmol/kg BIW for two weeks produced 43% partial response subjects.

### Example 11: In Vitro Studies of Conjugate 5 in Ovarian Cancer Cell Lines

### Reagents

The mouse and human folate binding protein 1 (FBP1, FOLR1) PicoKine™ ELSIA kits were purchased from Boster Biological Technology (Pleasanton, Calif.). Antibodies used for surface marker staining were purchased from eBioscience: PD-L1 (clone MIH5; cat # 25-5982), F4/80 (clone BM8; cat # 12-4801), CD11b (clone M1/70; cat # 48-0112), CD3c (clone 145-2C11; cat # 25-0031), CD4 (clone GK1.5; cat # 46-0041), and CD8β (clone H3517.2; cat # 11-0083).

### Cell Line

The FR-α expressing cell lines utilized to evaluate Conjugate 5 activity in in-vitro and ex-vivo studies were (1) ID8-C115, an ovarian carcinoma cell line transfected with the murine FR-α, and (2) IGROV1, a human ovarian carcinoma cell line that expresses the human FR-α. The FR-α negative ID8 parent (ID8p) cell line was used as controls in-vivo. ID8p and ID8-C115 cells were grown respectively in a folate-replete or folate-free RPMI1640 medium (Gibco BRL) (FFRPMI) containing 10% heat-inactivated fetal calf serum (HIFCS) and antibiotics, and maintained under a 5% CO₂ atmosphere using standard cell culture techniques. IGROV1 cells were grown in the same medium as ID8-C115 except that Corning® ultra-low attachment culture flasks (VWR, Cat. #89089-878) were used.

### ELISA Analysis

Following manufacturer's instructions, standards and test samples were added to 96-well ELISA plates that were pre-coated with a rat anti-FOLR1 monoclonal antibody. A biotinylated goat anti-FOLR1 polyclonal antibody was added and followed by a buffer wash. The avidin-biotin-peroxidase complex was then added and unbound conjugates were washed away. Subsequently, a horseradish peroxidase substrate, 3,3′,5,5′-Tetramethylbenzidine was added and catalyzed to produce a blue color product. The absorbance was read at 375 nm in a microplate reader at least two different time points.

### Clonogenic Assay

IGROV1 cells seeded in 6-well plates (1000 cells/well) were exposed for 2 hours to Conjugate 5 at 1, 10, and 100 nM and followed by a 9-day chase in drug-free medium. Afterwards, the cells were washed with PBS and fixed for 5 minutes in a 3:1 methanol:acetic acid solution. The cells were then stained with 0.5% crystal violet/methanol solution for 15 minutes and washed with tap water. After a drying step, the colonies were photographed and counted using the ImageJ software.

### Flow Cytometry

The single-cell suspensions prepared from ascites were blocked in a FACS stain solution on ice for 20 minutes prior to staining for flow cytometry. The FACS stain solution consisted of 1% bovine serum albumin fraction V (Fisher scientific, cat # BP1600), 0.5 mg/mL human immunoglobulin (Equitech-Bio, cat # SLH66) and 0.05% sodium azide in PBS. For surface marker detections (PD-L1, F4/80, CD11b, CD3, CD4, CD8), the tumor cells were stained in the FACS stain solution containing various fluorophore conjugated antibodies purchased from eBioscience at optimized concentrations (0.4-2.5 μg/mL). After 20 minutes on ice, the tumor cells were washed with PBS and re-suspended in PBS containing 3 μM propidium iodide for dead cell exclusion. Data was collected on the Gallios flow cytometer (Beckman Coulter) and analyzed using the Kaluza v1.2 software (Beckman Coulter). Functional folate receptor was measured using a small molecule synthesized in house by coupling folic acid to Alexa Fluor 647.

### Results

Conjugate 5 activity against ID8-C115 tumor cells was assessed using the XTT cell viability assay. The cells were exposed for 2 hours to 10-fold serial dilutions of Conjugate 5 (up to 1 μM) and followed by a 72-120 hours chase in drug-free medium. As determined by the XTT assay, Conjugate 5 showed a potent dose-dependent inhibition of cell proliferation with relative IC₅₀ values of ˜0.52 (72 h), 0.61 (96 h), and 0.17 (120 h) (FIG. 12). Importantly, the maximal cell kill was observed after 96-120 hours chase, supporting the mechanism of action of this class of DNA-crosslinking compound.

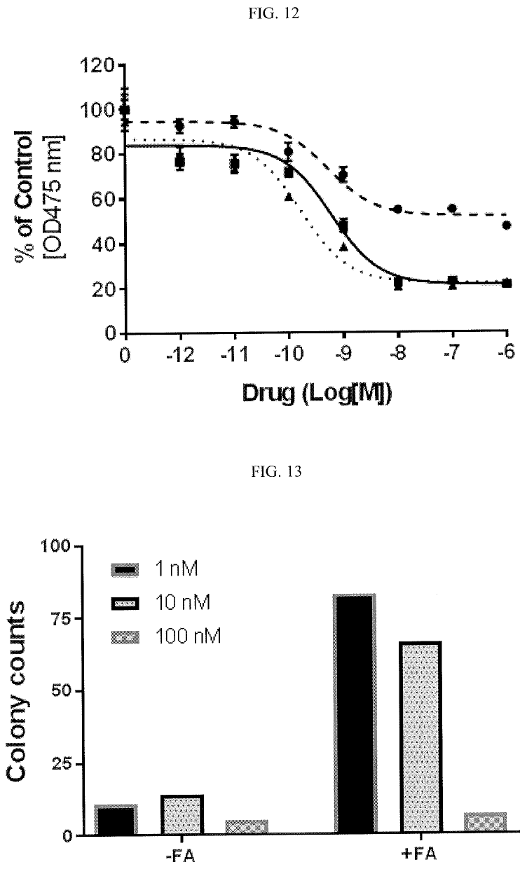

Conjugate 5 activity against the slow-growing IGROV tumor cells was assessed using a clonogenic assay. After a 2 hour exposure and 9-day chase (FIG. 13), Conjugate 5 demonstrated a potent activity at all concentrations (1-100 nM) tested. More importantly, Conjugate 5 anti-tumor activity was significantly reduced in the presence of excess amount of folic acid at both 1 and 10 nM concentrations.

### Example 12: In Vivo Studies of Conjugate 5 in Ovarian Tumor Model

### Mice

Female C57BL/6 (ID8p, ID8-C115) and nu/nu (IGROV1) mice were purchased from Envigo (Indianapolis, Ind.) and used when they reached 6-8 weeks of age. The mice were fed a folate-deficient diet (TestDiet, St. Louis, Mo.) on the day of arrival.

### Tumor Implantation

Mouse ascites tumors were generated by intra-peritoneal implantation of cultured cells at 5×10⁶ in C57BL/6 (ID8p, ID8-C115) and nu/nu (IGROV1) mice respectively.

Preparation of Single Cell Suspension from Tumor Bearing Mice

Ascites was collected via an I.P. injection of 5 mL of cold PBS containing 5 mM EDTA then removal of the intra-peritoneal fluid containing ascitic tumor cells. The cells were then collected by a 5 minute 400×g centrifugation, followed by an RBC lysis step, then a cold PBS wash and finally a 40 μm nylon filtration to remove tissue and large cellular aggregates.

Preparation of Acellular Ascitic Fluid from Ascites Bearing Mice

Upon euthanasia, total ascitic fluid was collected via an I.P. lavage of the intra-peritoneal fluid containing ascitic tumor cells. The acellular fraction of the ascitic fluid was obtained by a 5-minute 2200×g centrifugation and stored at −80° C. until future use.

### Conjugate 5 Plus Anti-CTLA-4 Combination Study

To test the effect of Conjugate 5 alone and in combination with anti-CTLA-4 antibody, ID8-C115 tumor cells (5×10⁶ cells per animal in 1% syngeneic mouse serum/folate-deficient RPMI1640 medium) were inoculated intraperitoneally 13 days post the date of arrival and start of the folate deficient diet. For comparison, EC1456 alone and in combination with the same regimen of anti-CTLA-4 antibody was also evaluated. Starting 7 days after tumor implant, mice were intravenously dosed BIW for a total of 6 doses with Conjugate 5 at 0.1 μmol/kg or EC1456 at 2 mol/kg. The anti-CTLA-4 antibody dosing solution was prepared by diluting the stock solution (BioXcell, Clone UC10-4F10-11) to 1.25 mg/mL in PBS, pH 7.4. Anti-CTLA-4 (250 μg/dose) was i.p. administered BIW for a total of 5 doses starting 11 days after the tumor implant. In the Conjugate 5 plus anti-CTLA-4 and EC1456 plus anti-CTLA-4 combination groups, all compounds were dose- and schedule-matched with the single-agent dosing groups. Mice were weighed 3 times/week and assessed for any clinical sign of swollen bellies indicative of ascites formation and for the evidence of toxicity such as respiratory distress, mobility, weight loss, diarrhea, hunched posture, and failure to eat. Once the animals developed ascites, they were monitored daily and euthanized when ascites became severe (rounded and walking on tip toes). Healthy animals from the same cohort of mice were used as controls for normal weight gain.

### Results

### Quantification of FBP1 in Mouse Ascitic Fluids

The acellular ascitic fluid samples collected from ID8p, ID8-C115 and IGROV1 tumor-bearing mice at the time of euthanasia were assayed for soluble murine (ID8p, ID8-C115) and human (IGROV1) FBP1 levels. Murine FBP1 was detected in the ascitic fluid derived from mice intraperitoneally implanted with ID8-C115 tumor cells at 0.93-4.6 nM (Table 1). Similarly, human FBP1 was detected in the ascitic fluid derived from mice intraperitoneally implanted with IGROV1 tumor cells at 0.70-2.8 nM (Table 1). In contract, negligible amount of the murine FBP1 was found in the ascitic fluid derived from ID8p tumor-bearing mice (Table 1). This suggests that malignant ascites microenvironment renders FOLR1 shedding from cancer cells.

### Assessment of Functional FR in Mouse Models of Ovarian Cancer

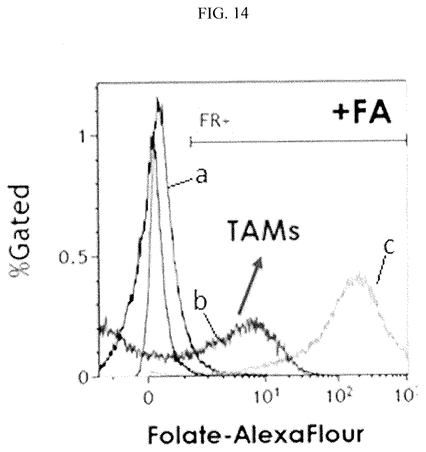

Functional FR levels were measured on the IGROV1 human ovarian cancer cells (FIG. 14; HLA+CD45−; label a) grown in the peritoneal cavity of nu/nu mice using a folate-fluorophore conjugate and compared to those on peritoneal macrophages (F480+CD11b+; label b) and freshly harvested IGROV1 cells from in vitro cultures (label c). There was only a small minority of mouse peritoneal ascites IGROV1 cells (˜6%) stained positive for FA-Alexa Fluor, suggesting a loss of FR-α either through shedding or down regulation or a combination of both. Shedding of FR-α by IGROV1 and ID8-C115 ascites cells likely occurred as soluble human and mouse FR-α (FBP1, FOLR1) were detected in acellular ascitic fluid by ELISA analysis (Table 1). The ID8p cell line derived ascitic fluid was used as a FRα-negative control and indeed very little soluble murine FR-α was detected by ELISA (Table 1).

TABLE 1

| Tumor models | Mouse strain | Ascites fluid | Results |
|---|---|---|---|
| (Intraperitoneal) | (Female) | ELISA analysis | (nM) |
| IGROV1 | Nu/Nu | hFBP1 | 0.70-2.8 |
| ID8-Cl15 | C57BL/6 | mFBP1 | 0.93-4.6 |
| ID8p_{(FRα- control)} | C57BL/6 | mFBP1 | 0.066-0.092 |

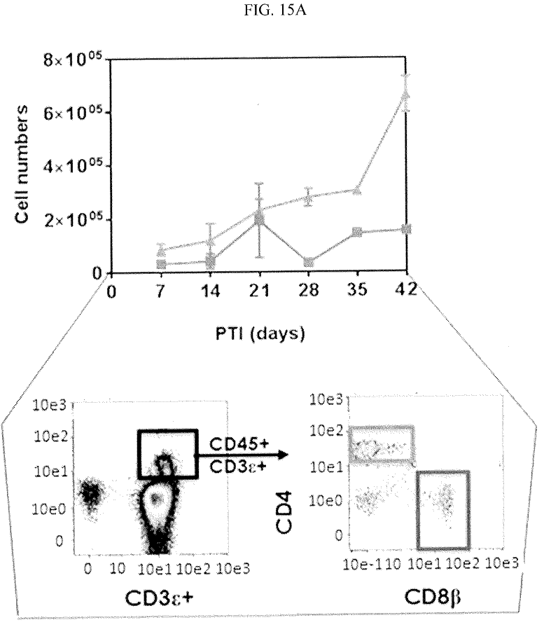

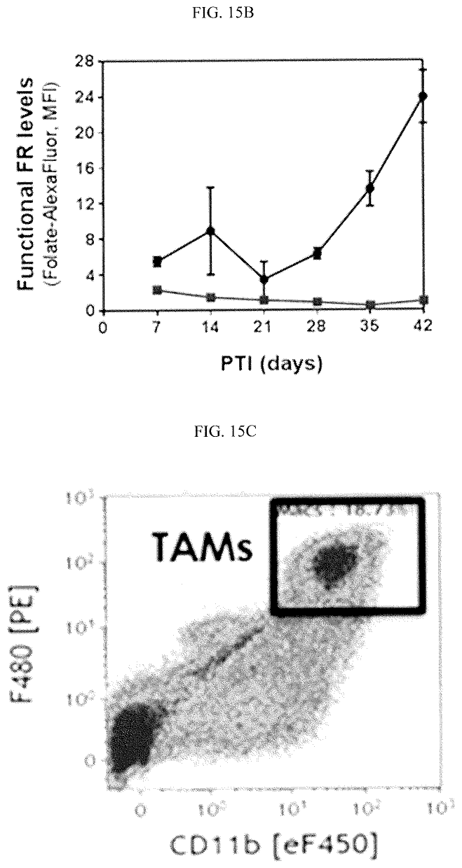

The presence of CD4+ and CD8+ T cells were also quantitated in total peritoneal cells of the immunocompetent C57BL6 mice at 7 day intervals post IP injection of the mouse ovarian cell line, ID8-CL15 (FIG. 15A). The CD45+CD3e+CD8+CD4− T cells (▪) slowly increased in number from day 7 to day 42 post implantation. The CD45+CD3e+CD4+CD8− T cells (▴) also increased in number from day 7 to day 35 with a more significant increase from day 35 to day 42 post implantation suggesting an immune response to the ovarian cancer cell had occurred. In addition, CD45-non bone-marrow derived ascites cells from ID8-CL15 implanted mice expressed very little functional FR (see FIG. 15B (▪)), whereas ascites macrophages (see FIG. 15B (●) and 15C (insert box)) expressed a significant amount of a functional FR (likely, FRβ). These suggest that targeting of FR-β+ ovarian cancer stromal cells such as ascites macrophages could be alterative mechanism of action for compounds such as Conjugate 5.

Conjugate 5 In-Vivo Activity Alone and in Combination with Anti-CTLA-4

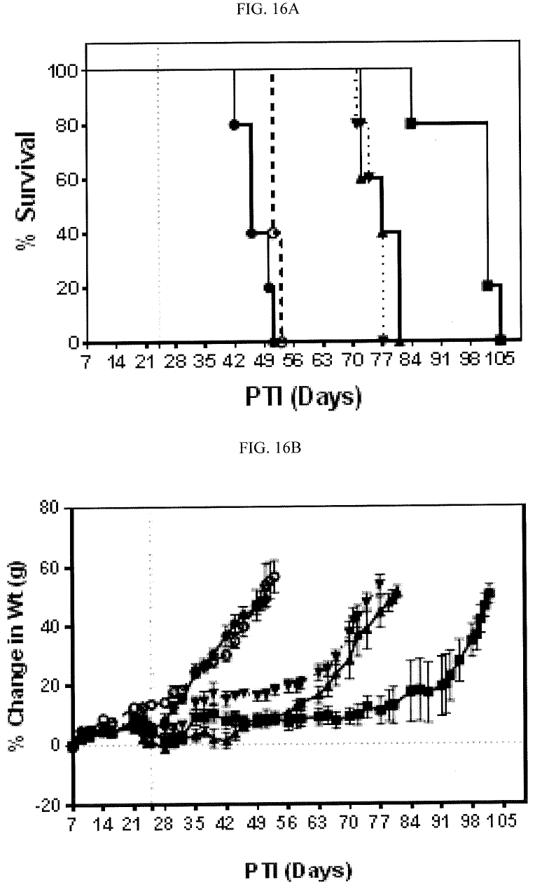

CTLA-4 (CD152) is a protein receptor that functions as an immune checkpoint to downregulate immune responses. CTLA-4 competes with CD28 for binding to B7 on antigen presentation cells in order to shut down T-cell activation. Recent studies showed that CTLA4 antagonists can enhance the activity of chemotherapy in certain tumor types. To examine the antitumor effect of Conjugate 5 alone and in combination anti-CTLA-4 antibody, we utilized syngeneic intraperitoneal ID8-C115 tumor bearing mice (FIG. 16A). For comparison, EC1456 was also tested as single agent or in combination with anti-CTLA-4 antibody. Here, untreated control mice had a median survival time of ˜46 days post tumor implant. Both EC1456 alone (i.v. 2 μmol/kg, BIW×6 doses) and Conjugate 5 alone (i.v. 0.1 μmol/kg, BIW×6 doses) produced significant anti-tumor effects in 5 animals each group, with ˜67% increase in the median survival time (˜77 days post tumor implant, P=0.0018, Log-Rank test). Anti-CTLA-4 antibody alone (i.p. 250 μg/dose, BIW×5 doses) displayed no significant anti-tumor effect in 5 animals, with ˜11% increase in the median survival time (˜51 days post tumor implant). EC1456 (i.v. 2 μmol/kg, BIW×6 doses) plus anti-CTLA-4 antibody (i.p. 250 μg/dose, BIW×5 doses) displayed no additional benefit in 5 animals with a median survival time of ˜81 days post tumor implant. On the other hand, Conjugate 5 (i.v. 0.1 mol/kg, BIW×6 doses) plus anti-CTLA-4 antibody (i.p. 250 μg/dose, BIW×5 doses), displayed additional therapeutic benefit in 5 animals with a median survival time of ˜102 days post tumor implant.

### Example 13: Comparison of Conjugate 5 and EC1456 Against Various Stages of ID8-C115 Ascites Tumor-Bearing Mice

### Materials

Conjugate 5 (M.W. 2369) and EC1456 (M.W. 2626) were synthesized in house.

### In-Vivo Methods

### Cell Line

ID8-C115 cells were grown in a folate-free RPMI1640 medium (Gibco BRL) (FFRPMI) containing 10% heat-inactivated fetal calf serum (HIFCS) and antibiotics, and maintained under a 5% CO₂ atmosphere using standard cell culture techniques.

### Mice

Female C57BL/6 mice were purchased from Envigo (Indianapolis, Ind.) and used when they reached 6-8 weeks of age. The mice were fed a folate-deficient diet (TestDiet, St. Louis, Mo.) on the day of arrival.

### Tumor Implantation

Mouse ascites tumors were generated by intra-peritoneal implantation of cultured cells at 5×10⁶ in C57BL/6 mice.

### Conjugate 5 Versus EC1456 Activity Against ID8-C115 Ascites Tumors In-Vivo

In a first experiment (P-1836), all treatment started 7 days after the tumors were implanted. The mice were intravenously dosed with Conjugate 5 at 0.1 μmol/kg twice-per-week for a total of 6 doses (BIW×3). For comparison, EC1456 was dosed at 2 μmol/kg twice-per-week for a total of 6 doses (BIW×3). In a second experiment (P-1846), Conjugate 5 treatment started 21 days after the tumors were implanted. The mice were intravenously dosed with Conjugate 5 at 0.1 μmol/kg for 3 consecutive days each week for 3 weeks (D0-2×3, 9 doses). For comparison, EC1456 was dosed at 2 μmol/kg for 3 consecutive days each week for 3 weeks (D0-2×3, 9 doses). In a third experiment (P-1861), Conjugate 5 treatment started 35 days after the tumors were implanted. The mice were intravenously dosed with Conjugate 5 at 0.3 μmol/kg once-a-week for 2 consecutive weeks (SIW×2, 2 doses). For comparison, EC1456 was dosed at 2 μmol/kg for 3 consecutive days each week for 2 weeks (D0-2×2, 6 doses). In a fourth experiment (P-1836), Conjugate 5 treatment started 43 days after the tumors were implanted. The mice were intravenously dosed with Conjugate 5 at 0.3 μmol/kg once-a-week for 2 consecutive weeks (SIW×2, 2 doses). Due to the advanced stage of the disease, EC1456 treated mice only received 2 μmol/kg for 3 consecutive days for 1 week (D0-2×1, 3 doses). All mice were weighed 3 times/week and assessed for any clinical sign of swollen bellies indicative of ascites formation and for evidence of toxicity including respiratory distress, mobility, weight loss, diarrhea, hunched posture, and failure to eat. Once the animals developed ascites, they were monitored daily and euthanized when ascites became severe (rounded and walking on tip toes).

### Data and Results

### Conjugate 5 Activity in 7-Day-Old ID8-C115 Ascites Tumor-Bearing Mice

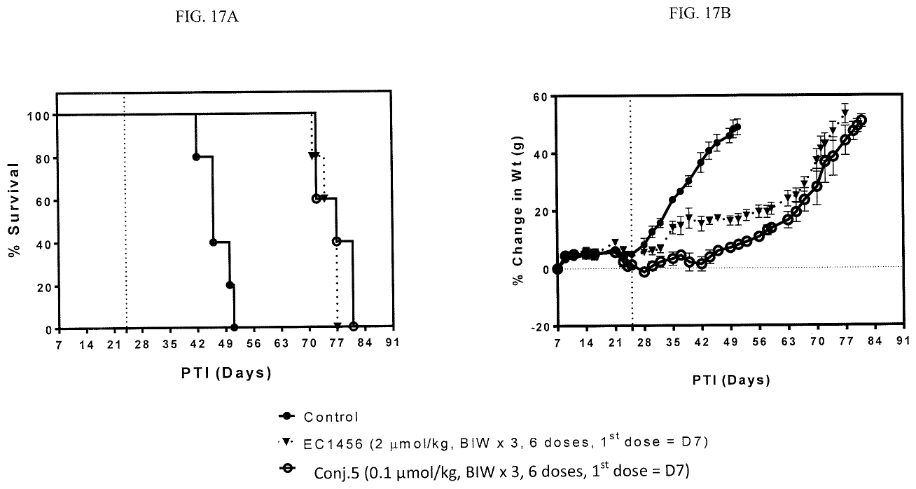

As shown in FIGS. 17A and 17B, untreated control mice had a median survival time of ˜46 days post tumor implant. Both Conjugate 5 (0.1 mol/kg, BIW×6 doses) and EC1456 (2 mol/kg, BIW×6 doses) produced similar anti-tumor effects in 5 animals in each group, with ˜67% increase in the median survival time (˜77 days post tumor implant).

### Conjugate 5 Activity in 21-Day-Old ID8-C115 Ascites Tumor-Bearing Mice

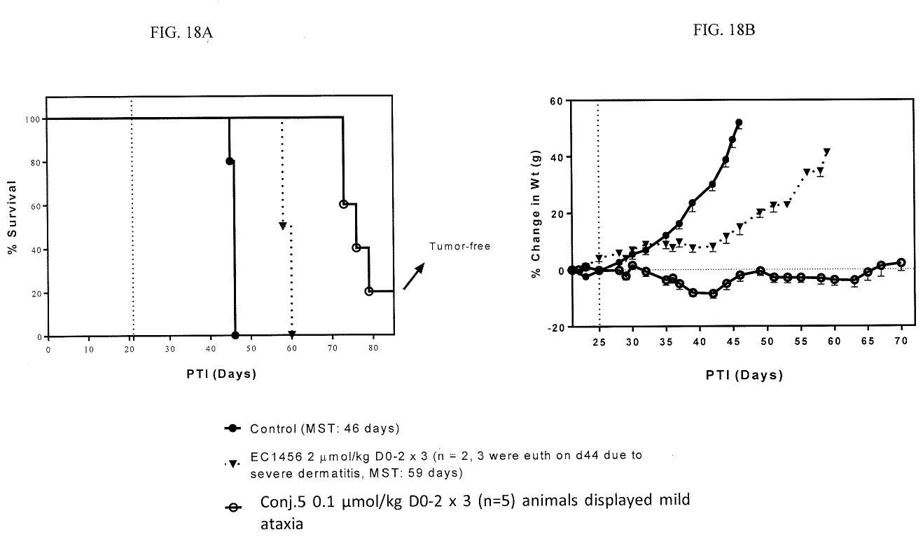

As shown in FIGS. 18A and 18B, untreated control mice had a median survival time of ˜46 days post tumor implant. Conjugate 5 (0.1 mol/kg, D0-2×3, 9 doses) produced a significant anti-tumor effect in 5 animals in each group, with ˜65% increase in the median survival time (˜76 days post tumor implant). Notably, all Conjugate 5 treated animals displayed mild ataxia at the end of study and one animal did not develop ascites (an outlier). EC1456 (2 μmol/kg, D0-2×, 9 doses) treated mice developed severe dermatitis and two animals were euthanized on Day 44 due to the skin condition. The two remaining animals developed ascites and had a median survival time of 59 days, ˜28% increase from the untreated controls.

### Conjugate 5 Activity in 35-Day-Old ID8-C115 Ascites Tumor-Bearing Mice

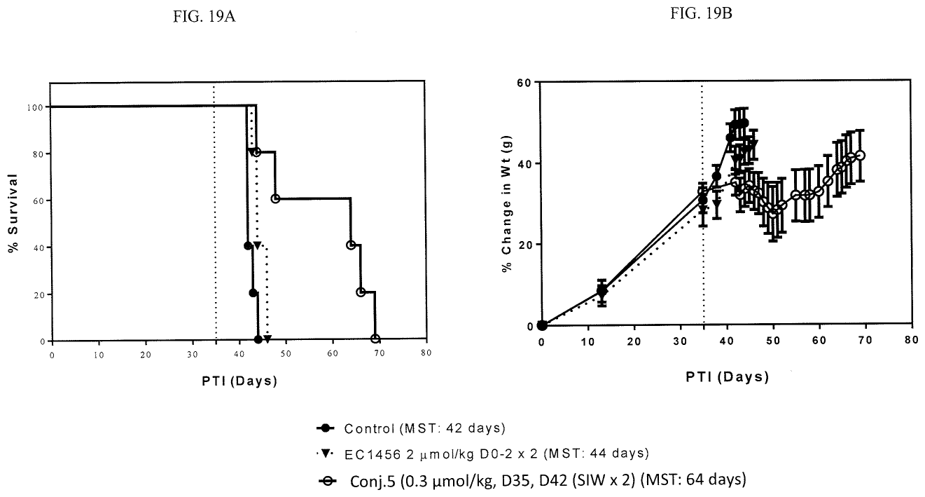

As shown in FIGS. 19A and 19B, untreated control mice had a median survival time of ˜42 days post tumor implant. Conjugate 5 (0.3 mol/kg, SIW×2, 2 doses) produced a significant anti-tumor effect in 5 animals in each group, with ˜52% increase in the median survival time (˜64 days post tumor implant). EC1456 (2 mol/kg, D0-2×2, 4 doses) produced no anti-tumor effects with a median survival time of ˜44 days post tumor implant, similar to that of untreated controls.

### Conjugate 5 Activity in 43-Day-Old ID8-C115 Ascites Tumor-Bearing Mice

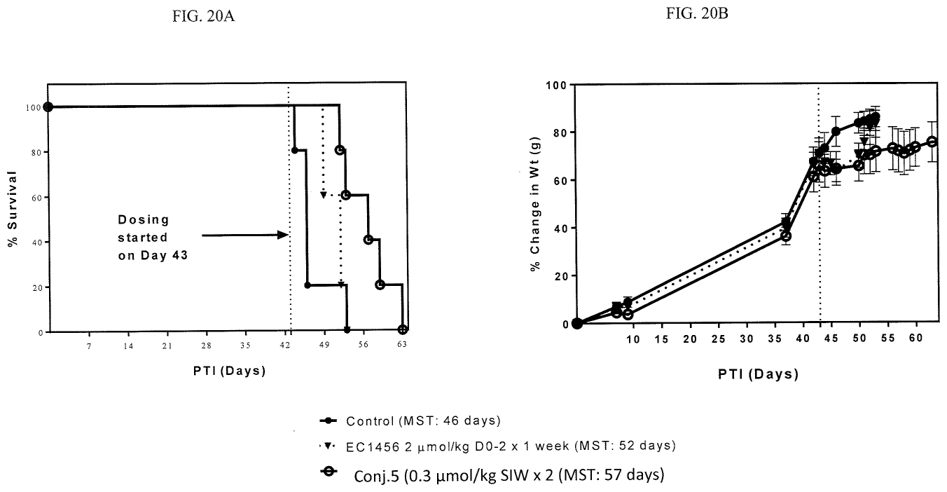

As shown in FIGS. 20A and 20B, untreated control mice had a median survival time of ˜46 days post tumor implant. Conjugate 5 (0.3 mol/kg, SIW×2, 2 doses) produced a significant anti-tumor effect in 5 animals in each group, with ˜24% increase in the median survival time (˜57 days post tumor implant). EC1456 (2 mol/kg, D0-2×1, 3 doses) produced ˜13% increase in median survival time (˜52 days post tumor implant) which was not significant from that of untreated controls.

### Comparison of Conjugate 5 and EC1456 Against Various Stages of ID8-C115.

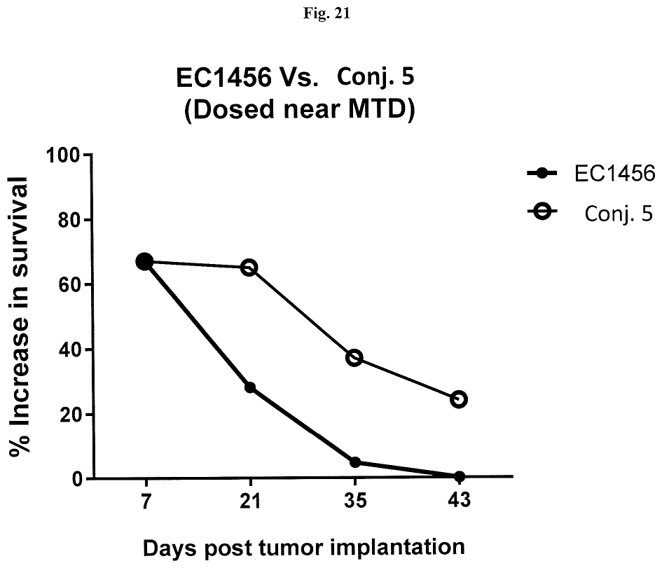

FIG. 21 summarizes the results of each experiment where ID8-C115 tumor-bearing mice at various stages of the disease were treated with Conjugate 5 and EC1456 at respective dosing regimens (some toxicity were observed as described above). However, as EC1456 gradually lost its strength in the advanced stages of ID8-C115 ascites tumor, Conjugate 5 was consistently more active. More importantly, from the onset of ascites (days 35) to end-stage of the disease that required euthanasia in untreated animals (day 43), Conjugate 5 provided a therapeutic benefit while EC1456 was completely inactive.

### Example 14: In-Vitro and In-Vivo Assays

### Materials

### Reagents

EC1456 (M.W. 2626) and Conjugate 5 (M.W. 2369) were synthesized in house. Antibodies used for surface marker staining were purchased from eBioscience: F4/80 (clone BM8; cat # 12-4801), CD11b (clone M1/70; cat # 48-0112).

In-Vitro Methods

### Cell Lines

The FRα− and FRα+ expressing cell lines utilized to evaluate Conjugate 5 activity in-vitro and/or ex-vivo studies were (1) 4T1p, a mouse breast cancer cell line that resembles triple negative breast cancer in humans, (2) 4T1-C12, 4T1p stably transfected with a mouse FRa, and (3) IGROV1, a human ovarian carcinoma cell line that expresses the human FRα. 4T1p and 4T1-C12 cells were grown respectively in a folate-replete or folate-free RPMI1640 medium (Gibco BRL) (FFRPMI) containing 10% heat-inactivated fetal calf serum (HIFCS) and antibiotics, and maintained under a 5% CO₂ atmosphere using standard cell culture techniques. IGROV1 cells were grown in the same medium as 4T1-C12.

### Cell Viability Assay

4T1p and 4T1-C12 tumor cells in 96-well plates (20,000 cells/well) were treated with 10-fold serial dilutions of Conjugate 5 (100 nM) in FFRPMI medium. After a 2 hour exposure, the drug-containing media were replaced and the cells were washed and allowed to incubate further for 96 hours. The cell viability was assessed by adding XTT (2,3-bis(2-methoxy-4-nitro-5-sulfo-phenyl)-2H-tetrazolium-5-carboxanilide) to the culture medium for 2 hours following the manufacturer's instructions. All results were expressed as % absorbance (minus background) relative to the untreated control cells.

### Clonogenic Assay

IGROV1 cells seeded in 6-well plates (1000 cells/well) were exposed for 2 hours to Conjugate 5 and EC1456 at 1, 10, 100, and 1000 nM and followed by a 9-day chase in drug-free medium. Afterwards, the cells were washed with PBS and fixed for 5 minutes in a 3:1 methanol:acetic acid solution. The cells were then stained with 0.5% crystal violet/methanol solution for 15 minutes and washed with tap water. After a drying step, the colonies were photographed and counted using the ImageJ software.

### Flow Cytometry

The single-cell suspensions prepared from 4T1p and 4T1-C12 tumors were blocked in a FACS stain solution on ice for 20 minutes prior to staining for flow cytometry. The FACS stain solution consisted of 1% bovine serum albumin fraction V (Fisher scientific, cat # BP1600), 0.5 mg/mL human immunoglobulin (Equitech-Bio, cat # SLH66) and 0.05% sodium azide in PBS. For surface marker detections (F4/80, CD11b), the tumor cells were stained in the FACS stain solution containing various fluorophore conjugated antibodies purchased from eBioscience at optimized concentrations (0.4-2.5 μg/mL). After 20 minutes on ice, the tumor cells were washed with PBS and re-suspended in PBS containing 3 μM propidium iodide for dead cell exclusion. Data was collected on the Gallios flow cytometer (Beckman Coulter) and analyzed using the Kaluza v1.2 software (Beckman Coulter).

### In-Vivo Methods

### Mice

Female Balb/c mice (4T1p, 4T1-C12) were purchased from Envigo (Indianapolis, Ind.) and used when they reached 6-8 weeks of age. The mice were fed a folate-deficient diet (TestDiet, St. Louis, Mo.) on the day of arrival.

### Tumor Implantation

Solid tumors in Balb/c mice were generated by subcutaneous implantation of cultured cells at 5×10⁵ (4T1p) and 2×10⁶ (4T1-C12) per animal in the mammary region.

Preparation of Single Cell Suspension from Tumor Bearing Mice

Tumor digestion solution was prepared by adding type IV collagenase (Sigma cat # C5138 at 0.5 mg/mL final), hyaluronidase (Sigma cat # H3506 at 0.5 mg/mL final) and DNase I (Sigma cat # DN25 at 0.1 mg/mL final) in serum and folate free RPMI1640 and was then warmed to 37C. Single cell preparations of 4T1 and 4T1-CL2 orthotopic tumors were prepared by excision of each tumor from the Balb/c mice and by washing in cold PBS. After the cold PBS wash, subcutaneous fat became visible on the surface of the excised tumors and was carefully peeled away prior to tumor digestion. After removal of the visible fat, the solid tumors were minced and incubated in 10 mL of tumor digestion solution for 1 hour at 37C with vigorous shaking. After digestion, the single cell preparation was pelleted down at 400× g for 5 minutes and supernatant was discarded. The pellet was treated with 5 mL of room temperature sterile 1×RBC lysis solution (VWR cat # 420301-BL) for 5 minutes to lyse any red blood cells. An equal volume of cold PBS was added to the solution and the tumor cells were pelleted again at 400×g for 5 minutes and the supernatant was discarded. The final pellet was resuspended in 10 mL of cold PBS then filtered using a 40 μm Falcon® Cell Strainers, Sterile, Corning (VWR cat # 21008-949) to remove any tissue debris and undigested tumor. The filtered cell solution was pelleted again and resuspended in FACS stain then fluorescently labeled antibodies were added for flow cytometry analysis.

### Conjugate 5 Single-Agent Activity In-Vivo

Starting on day 0, mice with mammary 4T1p (˜78.3±12.1 mm³) and 4T1-C12 (˜70.1±14.1 mm³) were scheduled to receive Conjugate 5 at 200 nmol/kg, biweekly for two weeks. 4T1p tumor bearing mice received a total of 3 doses only and 4T1-C12 tumor bearing mice received a total of 4 doses as planned. Mice were weighed and measured for tumor size 3 times a week. The tumor volumes were calculated by the following formula: V=0.5×a×b², where a is the longest axis across the tumor, and b is the shorter axis perpendicular to a. The animals were euthanized when the tumor volume reached ˜1500 mm³. Mice were also monitored closely for the evidence of toxicity such as respiratory distress, mobility, weight loss, diarrhea, hunched posture, and failure to eat. The last Conjugate 5 dose in the 4T1p tumor-bearing mice was skipped due to weight loss.

### Data and Results

### Conjugate 5 In-Vitro Activity Against 4T1-C12 and 4T1p Tumor Cells

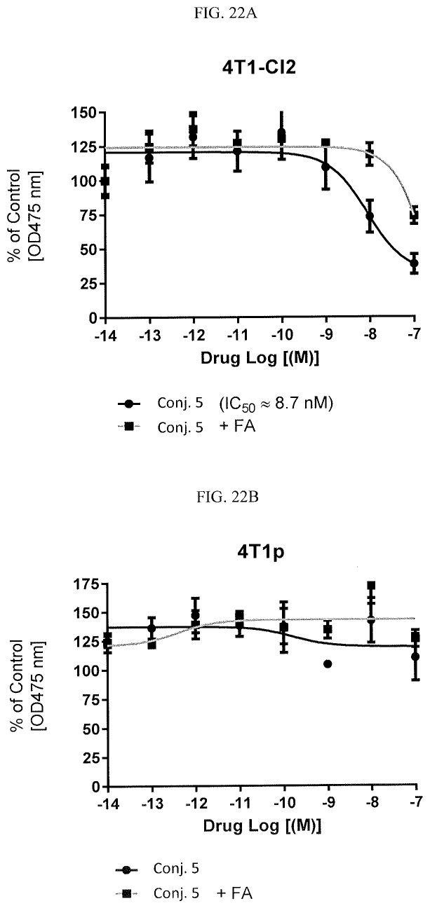

Conjugate 5 activity against 4T1-C12 and 4T1p tumor cells was assessed using the XTT cell viability assay. The cells were exposed for 2 hours to 10-fold serial dilutions of Conjugate 5 (up to 100 nM) and followed by a 96 hour chase in drug-free medium. In the FRα-positive 4T1-C12 tumor cell line, Conjugate 5 showed a dose-dependent inhibition of cell proliferation with a relative IC₅₀ value of ˜8.7 nM (FIGS. 22A and 22B). The activity against 4T1-C12 was partially reversible in the presence of excess folic acid under this testing condition. In comparison, Conjugate 5 was found completely inactive against the FRα-negative 4T1p tumor cell in-vitro.

### Conjugate 5 and EC1456 In-Vitro Activity Against Human IGROV Cells

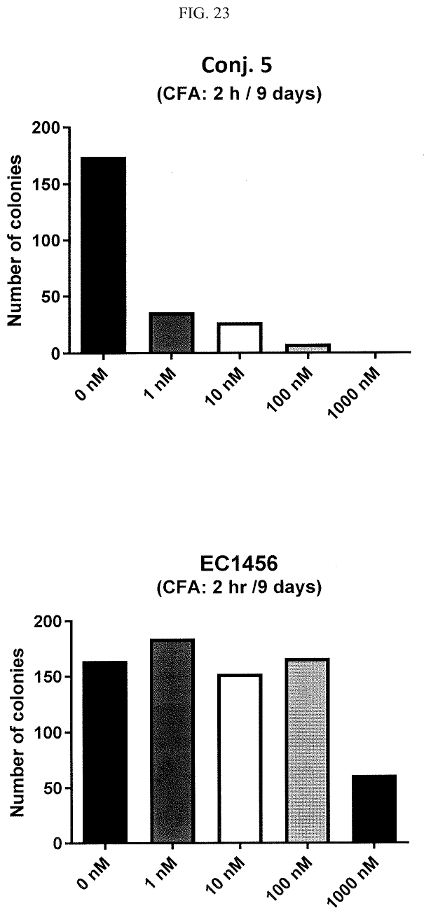

Conjugate 5 activity against the slow-growing IGROV tumor cells was compared against that of EC1456 in a standard clonogenic assay. After a 2 hour exposure and 9-day chase (FIG. 23), Conjugate 5 demonstrated a potent activity at all concentrations tested (1-1000 nM). On the other hand, significant EC1456 activity was only observed at 1 μM.

### Assessment of Tumor-Associated Macrophages in 4T1p and 4T1-C12 Tumors

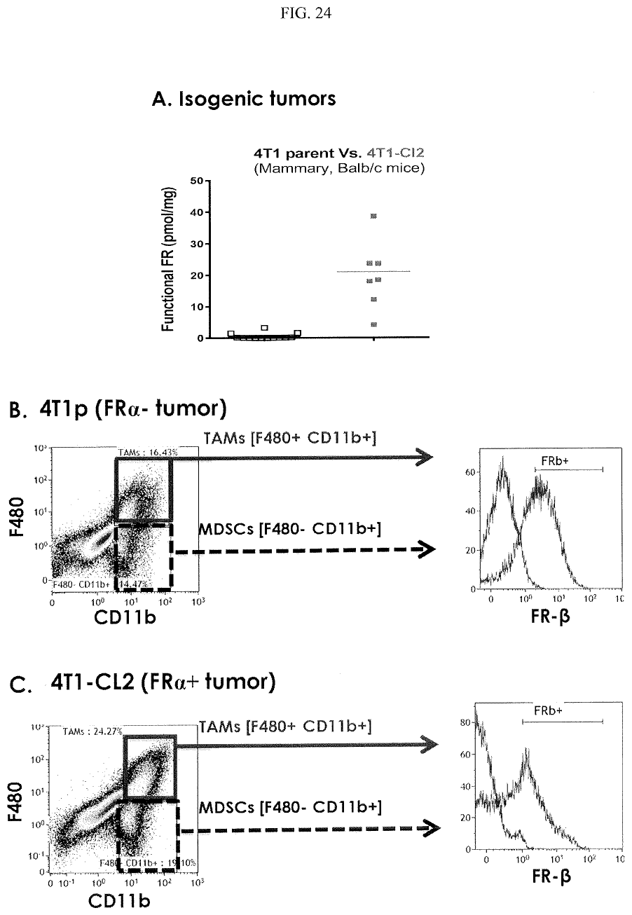

As shown in FIGS. 24A-C, orthotopic tumors (A) derived from the 4T1 mouse breast cancer cell line (open squares), possessed little detectable functional FR, while tumors grown from a FRα-transduced 4T1 subclone (4T1-C12; filled squares) contained significant levels. Tumor-associated macrophages (TAMs) found in 4T1 parent (B, ˜16%) and 4T1-C12 (C, ˜24%) tumors expressed FRβ while other non-macrophage myeloid cells (MDSCs) were FRβ-negative.

### Conjugate 5 In-Vivo Anti-TAM and Anti-Tumor Activity

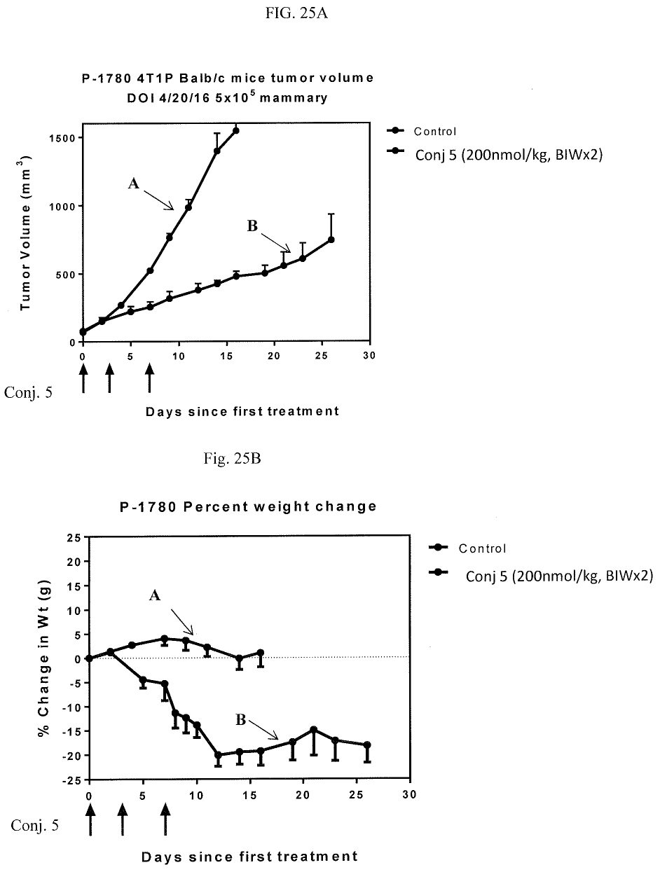

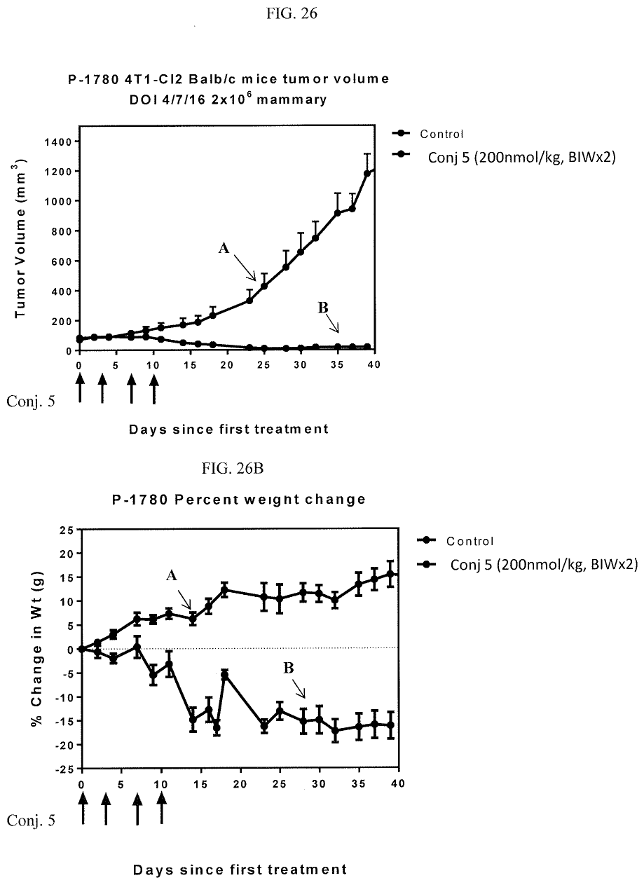

Conjugate 5 anti-TAM activity alone was assessed in the FRα-negative 4T1p tumor model. Conjugate 5 anti-tumor and anti-TAM dual activity was assessed in the FRα-positive 4T1-C12 tumor model. Flow cytometric analysis showed similar TAM content in both 4T1p and 4T1-C12 mammary tumors established in Balb/c mice. Despite the lack of activity in-vitro (FIG. 22A), 4T1p tumors showed a partial sensitivity to Conjugate 5 at 0.2 μmol/kg (i.v., BIW×3 doses) with a significant tumor growth delay (FIGS. 25A and 25B). However, there were no complete responders in this FRα-negative model. On the other hand, Conjugate 5 at 0.2 μmol/kg (i.v., BIW×4 doses) produced 3 out 5 complete responders in the FRα-positive 4T1-C12 tumor model (FIGS. 26A and 26B). In both cases, Conjugate 5 treatment caused significant weight loss in animals. But the data suggested that Conjugate 5 activity against FRα-positive tumor models could be enhanced by the presence of FRβ-positive TAMs.

### Example 15: Conjugate 5 Activity Against 4T1 TAMs In-Vivo

Female Foxn1^{nu} nude rats (Harlan, Inc., Indianapolis, Ind.) on a folate-deficient diet were subcutaneously implanted with 1×10⁶ 4 T1 tumor cells in the mammary region. When the tumors reached ˜1088 mm³, the animals (n=3) were intravenously dosed with nothing (Control), 254 nmol/kg of Conjugate 5, 254 nmol/kg of Conjugate 5 plus 127 μmol/kg of EC0923, or 127 μmol/kg of EC0923. Four days later, the entire tumors were harvested, enzymatically digested, and subjected to FACS analysis. The tumor cell suspensions were stained for macrophage markers (CD163, CD11b), cell viability (propidium iodide), and late and early apoptosis (Annexin V).

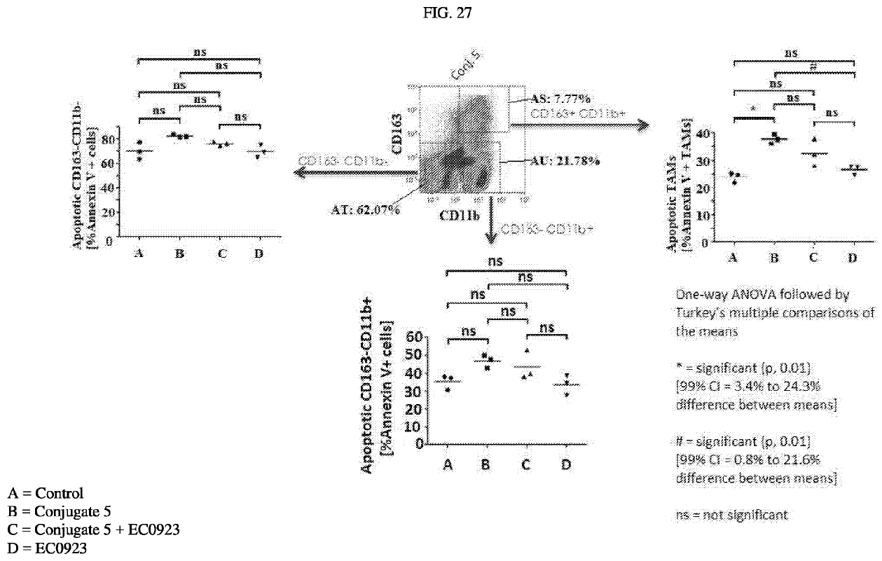

Conjugate 5 demonstrated in-vivo selectivity for FR+ 4T1 TAMs over FR− 4T1 tumor cells (FIG. 27). With a single administration, Conjugate 5 was shown to significantly decrease the CD163+CD11b+ TAM population in these 4T1 tumors. While the folate competitor EC0923 (a folate not linked to a drug) alone did not have any effect on 4T1 TAMs, Conjugate 5 anti-TAM activity was also not blocked by the 500-fold excess of EC0923. Further analysis revealed that Conjugate 5 had no effect against FR− cell populations including CD163-CD11b+4T1 TAMs and 4T1 tumor cell themselves. These data show that the maximum apoptosis (killing) of TAMs occurred with Conjugate 5 treatment.

1. (canceled)

2. A method for treating a folate receptor negative cancer comprising administering to the host animal a therapeutically effective amount of Conjugate 5, or a pharmaceutically acceptable salt thereof, to deplete tumor-associated macrophages.

3. A method for treating a folate receptor negative cancer comprising administering to the host animal a therapeutically effective amount of Conjugate 5, or a pharmaceutically acceptable salt thereof, and treating the folate receptor negative cancer having tumor-associated macrophages.

4. A method for treating a folate receptor negative cancer in a host animal comprising administering to the host animal a therapeutically effective amount of Conjugate 5, or a pharmaceutically acceptable salt thereof, to target tumor associated macrophages.

5.-8. (canceled)

9. The method of claim 2 wherein tumor associated macrophages are in the cancer and the tumor-associated macrophages have the pro-tumor M2-biased CD163(+) phenotype.

10. The method of claim 2 wherein tumor-associated macrophages are in the cancer and the tumor-associated macrophages have the pro-tumor M2-biased CD163(+) and TGF-β(+) phenotype.

11. The method of claim 2 wherein tumor-associated macrophages are in the cancer and the tumor-associated macrophages have the pro-tumor M2-biased CD11b(+) phenotype.

12. The method of claim 2 wherein tumor-associated macrophages are in the cancer and the tumor-associated macrophages have the pro-tumor M2-biased CD163(+) and CD11b(+) phenotype.

13. The method of claim 2 wherein tumor-associated macrophages are in the cancer and the tumor-associated macrophages have the pro-tumor M2-biased F480(+) phenotype.

14. The method of claim 2 wherein tumor-associated macrophages are in the cancer and the tumor-associated macrophages have the pro-tumor M2-biased F480(+) and CD11b(+) phenotype.

15.-16. (canceled)

17. The method of claim 2 wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkin's lymphoma, uveal melanoma, glioblastoma, renal carcinoma, leiomyosarcoma, and pigmented villonodular synovitis.

18.-25. (canceled)

26. The method of claim 2 wherein tumor-associated macrophages are in the cancer and the tumor-associated macrophages have the pro-tumor M2-biased CD163(+), IL10(+), Arg1(+), TGF-β(+), VEGF(+), CD206(+), CD11b(+), and F480(+) phenotype.

27. The method of claim 3 wherein tumor associated macrophages are in the cancer and the tumor-associated macrophages have the pro-tumor M2-biased CD163(+) phenotype.

28. The method of claim 4 wherein tumor associated macrophages are in the cancer and the tumor-associated macrophages have the pro-tumor M2-biased CD163(+) phenotype.

29. The method of claim 3 wherein tumor-associated macrophages are in the cancer and/or form part of the tissue or cancer and the tumor-associated macrophages are pro-tumor M2-biased and express one or more markers selected from the group consisting of CD163(+), IL10(+), Arg1(+), TGF-β(+), VEGF(+), CD206(+), CD11b(+), and F480(+) phenotype.

30. The method of claim 4 wherein tumor-associated macrophages are in the cancer and/or form part of the tissue or cancer and the tumor-associated macrophages are pro-tumor M2-biased and express one or more markers selected from the group consisting of CD163(+), IL10(+), Arg1(+), TGF-β(+), VEGF(+), CD206(+), CD11b(+), and F480(+) phenotype.

31. The method of claim 3 wherein tumor-associated macrophages are in the cancer and the tumor-associated macrophages have the pro-tumor M2-biased CD163(+), IL10(+), Arg1(+), TGF-β(+), VEGF(+), CD206(+), CD11b(+), and F480(+) phenotype.

32. The method of claim 4 wherein tumor-associated macrophages are in the cancer and the tumor-associated macrophages have the pro-tumor M2-biased CD163(+), IL10(+), Arg1(+), TGF-β(+), VEGF(+), CD206(+), CD11b(+), and F480(+) phenotype.

33. The method of claim 3 wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkin's lymphoma, uveal melanoma, glioblastoma, renal carcinoma, leiomyosarcoma, and pigmented villonodular synovitis.

34. The method of claim 4 wherein the cancer is selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkin's lymphoma, uveal melanoma, glioblastoma, renal carcinoma, leiomyosarcoma, and pigmented villonodular synovitis.
